(19) United States
(12) Patent Application Publication
(10) Pub. No.: US 2010/0226875 A1
(43) Pub. Date: Sep. 9, 2010

(54) CONTROLLED RELEASE OF ACTIVE COMPOUNDS FROM DYNAMIC MIXTURES

(76) Inventors: Andreas Herrmann, Veyrier (CH); Jean-Marie Lehn, Strasbourg (FR); Guillaume Godin, Collonges (FR)

Correspondence Address: WINSTON & STRAWN LLP;PATENT DEPARTMENT, 1700 K STREET, N.W., WASHINGTON, DC, 20006, US

(21) Appl. No.: 12/161,780

(22) PCT Filed: Jan. 19, 2007
(86) PCT No.: PCT/IB07/50187

§ 371 Date: Jul. 22, 2008

(30) Foreign Application Priority Data
Jan. 24, 2006 (EP) 06100755.5
Oct. 17, 2006 (EP) 06122424.2

(51) Int. Cl. A61K 8/33; A61Q 13/00; A01N 35/00; A23L 1/221; A61K 8/35

(52) U.S. Cl. 424/84; 512/27; 514/675; 514/693; 426/650

(57) ABSTRACT

The present invention relates to a dynamic mixture in the form of a dynamic mixture obtained by reacting together, in the presence of water, at least one O-substituted hydroxylamine or S-substituted thiohydroxylamine derivative with at least one perfuming, flavoring, insect repellent or attractant, bactericide or fungicide aldehyde or ketone. This mixture is capable of releasing in a controlled and prolonged manner the aldehyde or ketone into the surrounding environment. Furthermore, the present invention concerns also the use of these dynamic mixtures as perfuming ingredients as well as the perfuming compositions or perfumed articles that include these mixtures.

### TECHNICAL FIELD

The present invention concerns a dynamic mixture obtained by combining, in the presence of water, at least one O-substituted hydroxylamine(alkoxyamine) or S-substituted thiohydroxylamine derivative of formula (I), as defined further below, with at least one active aldehyde or ketone. The invention's mixture is capable of releasing in a controlled and prolonged manner said active compound in the surrounding environment.

The present invention concerns also the use of said dynamic mixtures as perfuming ingredients as well as the perfuming compositions or perfumed articles comprising the invention's mixtures. A further object of the present invention is the use of specific O-substituted hydroxylamine or S-substituted thiohydroxylamine derivatives as additives to prolong the perfuming effect of particular aldehydes or ketones.

### PRIOR ART

Active compounds, such as fragrances, but also insect attractants or repellents, as well as some bactericides, are volatile molecules that can only be perceived over a limited period of time.

The perfume industry has a particular interest for compositions or additives which are capable of prolonging or enhancing the perfuming effect of a mixture of several perfuming ingredients at the same time over a certain period of time. It is particularly desirable to obtain long-lasting properties for standard perfumery raw materials which are too volatile or have a poor substantivity by themselves, or which are only deposited in a small amount onto the surface of the final application. Furthermore, some of the perfumery ingredients, especially aldehydes, are unstable and need to be protected against slow degradation prior to their use. Long-lasting perfumes are desired for various applications, as for example fine or functional perfumery or cosmetic preparations. The washing of textiles is a particular field in which there is a constant quest to enable the effect of active substances, in particular perfumes, to be effective for a certain period of time after washing and drying. Indeed, many substances having odors which are particularly suitable for this type of application are known to lack tenacity on laundry, or do not remain on the laundry when rinsed, with the result that their perfuming effect is experienced only briefly and not very intensely. Given the importance of this type of application in the perfume industry, research in this field has been sustained, in particular with the aim of finding new, and more effective solutions to the aforementioned problems.

A variety of precursor molecules which release active material by a chemical reaction during or after application (using O₂, light, enzymes, water (pH) or temperature as the release trigger) have been described as an alternative to encapsulation systems. In general, due to their inherent instability, the precursors often decompose in the application base during storage and thus release their fragrance raw material before the desired use.

Dynamic libraries using derivatives of formula (I) are known from the pharmaceutical industry. However, in such prior art libraries, the aldehydes or ketones are pharmaceutically active compounds, i.e. chemicals not intended to be volatiles, and the libraries themselves are either used to generate a multitude of more or less biologically active compounds or for the rapid generation and identification of biological receptors or ligands.

None of the prior art documents suggests, or allows to reasonably expect that the reversibility of the formation of addition products between carbonyl compounds and derivatives of formula (I) may allow to deliver said carbonyl compounds in a controlled manner or that the dynamic mixtures thus obtained can be used successfully as perfuming ingredients or even that they allow to prolong the fragrancing effect of a perfuming compound, especially in a consumer product.

To the best of our knowledge, none of the compositions of the present invention has been described for the controlled delivery of standard (i.e. of current use) perfumery ketones or aldehydes. Some of said compositions have been described as such in the context of the synthesis of specific oximes (see EP 0 672 746). However, in said patent document the compositions have been used to isolate the pure oxime, i.e. they have not been used for the delivery of carbonyl compounds, and have not been suggested for such use. Indeed it is the oxime, as such, and not the reaction mixture, which is used as perfuming ingredient showing its own olfactive note and not the ones of the carbonyl compound derived from.

### DESCRIPTION OF THE INVENTION

We have now surprisingly found that a dynamic mixture, obtainable by combining, in the presence of water, at least one derivative of formula (I) with at least one active aldehyde or ketone (O═CR₂) is a valuable ingredient capable of releasing, in a controlled and prolonged manner, said active aldehyde or ketone.

As “dynamic mixture” we mean here a composition comprising a solvent and several starting components as well as several addition products that are the results of reversible reactions between the various starting components. Said dynamic mixtures take advantage from reversible chemical reactions, in particular from the formation and dissociation by reversible condensation between the carbonyl group of the active aldehyde or ketone and the NH₂ moiety of the derivative of formula (I). The ratio between the various starting and addition products depends on the equilibrium constant of each possible reaction between the starting components. The usefulness of said “dynamic mixture” derives from a synergistic effect between all the components.

By the term “active” we mean here that the aldehyde or ketone to which it is referred is capable of bringing a benefit or effect into its surrounding environment, and in particular a perfuming, flavoring, insect repellent or attractant, bactericide and/or fungicide effect. Therefore, for example, said “active aldehyde or ketone” possesses at least one property which renders it useful as perfuming or flavoring ingredient, as insect repellent or attractant or as bactericide or fungicide.

According to all the above and below mentioned embodiments of the invention, the invention's dynamic mixture is particularly useful when the active aldehyde or ketone is a perfuming ingredient, i.e. a perfuming aldehyde or ketone. A “perfuming aldehyde or ketone” is a compound, which is of current use in the perfumery industry, i.e. a compound which is used as active ingredient in perfuming preparations or compositions in order to impart a hedonic effect. In other words, such an aldehyde or ketone, to be considered as being a perfuming one, must be recognized by a person skilled in the art of perfumery as being able to impart or modify in a positive or pleasant way the odor of a composition, and not just as having an odor. From now on we will refer to said “perfuming aldehyde or ketone” also as “perfuming compounds”.

The invention is carried out exactly in the same manner, independently of the exact properties of the active aldehyde or ketone. Therefore, it is understood that, even if the invention will be further illustrated hereinbelow with a specific reference to “perfuming compounds”, the below embodiments are also applicable to other active aldehydes or ketones (i.e. it is possible to replace the expression “perfuming” with “insect attractant”, “insect repellent” or with “bactericide”, for instance).

As previously mentioned, the invention's dynamic mixture enables a controlled release of an active aldehyde or ketone, and in particular a perfuming one. Such a behavior makes the invention's dynamic mixture particularly suitable as active ingredient. Consequently, the use of an invention's dynamic mixture as active ingredient is an object of the present invention. In particular it concerns a method to confer, enhance, improve or modify the odor properties of a perfuming composition or of a perfumed article, which method comprises adding to said composition or article an effective amount of an invention's dynamic mixture.

Now, according to a particular embodiment of the invention, the present invention concerns a use as perfuming ingredient of a dynamic mixture, for the controlled release of active ketones or aldehydes, obtainable by reacting, in a water-containing medium,

i) at least one derivative of formula

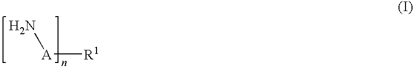

wherein:
A represents a functional group selected from the group consisting of the formulae (i) to (vi)

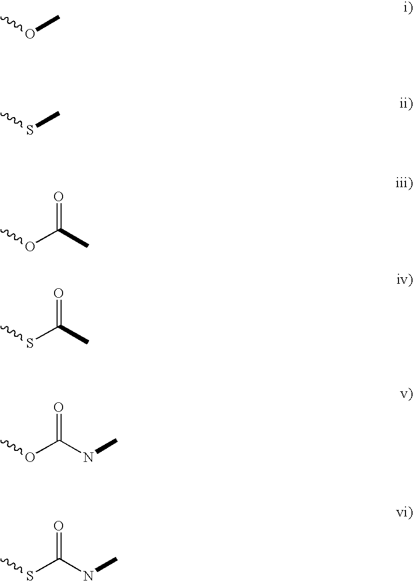

in which formulae the wavy line indicates the location of the bond between said NH₂ group and A, the bold lines indicate the location of the bond between said A and R¹; and
I) n is an integer varying from 1 to 4; and
   R¹ represents a trityl group or a mono-, di-, tri- or tetra-radical derived from a C₁ to C₁₈ linear, branched, cyclic or multicyclic hydrocarbon or polyethylene- or polypropylene-glycol group; said R¹ optionally comprising one, two, or three functional groups selected from the group consisting of silyl, ketone, amide, ether, thioether or secondary or tertiary amines; said R¹ being optionally substituted by up to three groups selected from the group consisting of NR²₂, NHR², (NR²R³₂)X, OR³, SO₃M, R³ and COOR⁴, R² representing a C₁ to C₁₀ alkyl, acyl or polyethylene- or polypropylene-glycol group, a phenyl group or a C₆ to C₉ alkylaryl group, and said R³ representing a hydrogen atom or a R² group, M representing a hydrogen atom or an alkali metal ion, R⁴ representing a M group or a R³ group and X representing a halogen atom or a sulphate; or
II) n is an integer varying from 2 to 5000; and R¹ represents a polyalkylene chain comprising between 2 and 5000 monomeric units;
   with
   ii) at least one active aldehyde or ketone having a molecular weight comprised between 80 and 230 g/mol and being a perfuming, flavoring, insect repellent or attractant, bactericide or fungicide ingredient, in particular being selected from the group consisting of the C₅₋₂₀ perfuming aldehydes and the C₅₋₂₀ perfuming ketones.

The dynamic mixture is obtained by reacting one or more derivatives of formula (I) with one or more perfuming ingredients in a water-containing medium. By “water-containing medium” we mean here a dispersing medium comprising at least 10% w/w, or even 30% w/w, of water and optionally an aliphatic alcohol such as a C₁ to C₃ alcohol, for example ethanol. More preferably, said medium comprises at least 50% w/w, or even 70%, water optionally containing up to 30% of a surfactant. According to a particular embodiment of the invention, the water-containing medium may have a pH comprised between 2 and 6.

According to another particular embodiment of the invention, the preferred derivatives of formula (I) are those wherein:
A represents a functional group selected from the group of the formulae (i) or (ii), as defined above; and
I) n is an integer varying from 1 to 4; and
   R¹ represents a trityl group or a mono-, di-, tri- or tetra-radical derived from a phenyl, benzyl or trityl group or from a C₁ to C₁₂ linear, branched, cyclic or bicyclic hydrocarbon or polyethylene- or polypropylene-glycol group, or a C₃-C₉ trialkyl amine; said R¹ optionally comprising one, two, or three functional groups selected from the group consisting of silyl, ketone, amide, ether, thioether or secondary or tertiary amines; said R¹ being optionally substituted by up to three groups selected from the group consisting of NR²₂, NHR², (NR²R³₂)X, OR³, SO₃M, R³ and COOR⁴, R² representing a C₁ to C₁₀ alkyl, acyl or polyethylene- or polypropylene-glycol group, a phenyl group or a C₆ to C₉ alkylaryl group, and said R³ representing a hydrogen atom or a R² group, M representing a hydrogen atom or an alkali metal ion, R⁴ representing a M group or a R³ group and X representing a halogen atom or a sulphate; or
II) n is an integer varying from 2 to 5000; and R¹ represents a polyalkylene chain comprising between 2 and 5000 monomeric units.

Alternatively, according to a further invention embodiment derivative of formula (I) is a compound of formula

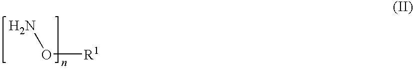

wherein
I) n is an integer varying from 1 to 4; and
   R¹ represents a trityl group or a mono-, di-, tri- or tetra-radical derived from a phenyl, benzyl or trityl group or from a C₁ to C₁₂ linear, branched, cyclic or bicyclic hydrocarbon or polyethylene- or polypropylene-glycol group, or a C₃-C₉ trialkyl amine; said R¹ optionally comprising one, two, or three functional groups selected from the group consisting of silyl, ketone, amide, ether, thioether or secondary or tertiary amines; said R¹ being optionally substituted by up to three groups selected from the group consisting of NR²₂, NHR², (NR²R³₂)X, OR³, SO₃M, R³ and COOR⁴, R² representing a C₁ to C₁₀ alkyl, acyl or polyethylene- or polypropylene-glycol group, a phenyl group or a C₆ to C₉ alkylaryl group, and said R³ representing a hydrogen atom or a R² group, M representing a hydrogen atom or an alkali metal ion, R⁴ representing a M group or a R³ group and X representing a halogen atom or a sulphate; or
II) n is an integer varying from 2 to 5000; and R¹ represents a polyalkylene chain having a molecular weight comprised between 48 and 80000.

In all the embodiments of the invention, by “polyalkylene chain” we mean here a chain which is derived by the polymerization of a monomer or several co-monomers comprising the moiety of formula —R′C═C(R′)₂, each R′ representing a hydrogen atom or a C₁₋₇ group chain such as a C₁₋₃ alkyl or even a phenyl group.

In all the embodiments of the invention, by “polyethylene- or polypropylene-glycol group” we mean here a chain of formula —(OCH₂CHR″)ₜ—OR′″, t representing an integer from 1 to 9, each R″ representing a hydrogen atom or a methyl group and R′″ representing a hydrogen atom or a hydrocarbon group.

More specifically, as non-limiting examples of O-substituted hydroxylamine or S-substituted thiohydroxylamine derivatives in the above-mentioned embodiments, one may cite the following classes:
i) Ar(CO)ₐONH₂, Ar—NH—CO—ONH₂, Ar(CO)ₐSNH₂ or Ar—NH—CO—SNH₂, wherein a is 1 or 0 and Ar is a substituted or non-substituted C₆₋₉ phenyl group, such as phenyl or tolyl or C₆H₄COOH;
ii) Alk(CO)ₐONH₂, Alk-NH—CO—ONH₂, Alk(CO)ₐSNH₂ or Alk-NH—CO—SNH₂, wherein a is 1 or 0 and Alk is a C₁-C₁₈ linear, branched or cyclic hydrocarbon group, preferably alkyl, optionally substituted by two OH groups, such as a C₁, C₂, C₃, C₄, C₅, C₆, C₈, C₁₂, C₁₆, C₁₈, (CHOH)₂ or CH₂(CHOH)₂CH₂ group;
iii) (H₂NO(CO)ₐ)ₛ-Alk or (H₂NS(CO)ₐ)ₛ-Alk, wherein a and Alk are as defined above and s is 2, 3 or 4;
iv) H₂NO(CO)ₐ—(CH₂)ₚ-T, wherein a is defined as above, p is an integer varying from 1 to 12 and T is a COOM, COOR⁵, SO₃M or N(R⁵)₃X group, M represents a Na or K cation, X represents a Cl or sulphate anion and R⁵ represents a C₁-C₄ alkyl group;
v) H₂NO(COCH₂O)ₐ—(CH₂CH₂O)_{q}—U or H₂NO(COCH₂O)ₐ—(CH₂CH₂O)_{q}—(CH₂CO)ₐONH₂ wherein a is defined as above, q is an integer varying from 1 to 9 and U is a hydrogen atom or a CH₃ group;
vi) H₂NS(COCH₂O)ₐ—(CH₂CH₂O)_{q}—U or H₂NS(COCH₂O)ₐ—(CH₂CH₂O)_{q}—(CH₂CO)ₐSNH₂ wherein a is defined as above, q is an integer varying from 1 to 9 and U is a hydrogen atom or a CH₃ group;
vii) (H₂NO(CO)ₐ(CH₂)₂₋ₐ)ᵣ(R⁶)₂₋ᵣ, NCH₂CH₂N(R⁶)₂₋ᵣ(CH₂)₂₋ₐ(CO)ₐONH₂)ᵣ or (H₂NO(CO)ₐ(CH₂)₂₋ₐ)ₘ(R⁶)₃₋ₘN, wherein a is defined as above, r is 1 or 2, m is 1, 2 or 3 and R⁶ is a hydrogen atom or a C₁-C₄ alkyl group;
viii) Q((CH₂)ₐCOOR⁶)₃₋ₘ((CH₂)ₐ(CO)ₐONH₂)ₘ, wherein R⁶ is as defined above, m is 1, 2 or 3 and Q is N or COR⁶, a being as defined above;
ix) Ph₃CONH₂, wherein Ph stands for phenyl;
x) R⁵₃Si(CH₂)ₙONH₂, wherein R⁵ and n are as defined above, or
xi) an O-substituted poly-hydroxylamine or an S-substituted poly-thiohydroxylamine derivative of poly(methyl methacrylate) and co-polymers thereof, of poly(methyl acrylate) and co-polymers thereof or of poly(4-vinylbenzoates) and co-polymers thereof.

In particular, there can be used the above-mentioned derivatives of O-substituted hydroxylamine.

Further specific examples of O-substituted hydroxylamine are the following compounds:
H₂NO—(CH₂CH₂O)_{q}-Me or H₂NO—(CH₂CH₂O)_{q}—CH₂CH₂ONH₂, wherein q is an integer varying from 1 to 4, such as MeOCH₂CH₂ONH₂;
H₂NOCH₂CH₂OCH₂CH₂OCH₂CH₂OMe or H₂NOCH₂CH₂OCH₂CH₂OCH₂CH₂ONH₂;
H₂NOCH₂COOR⁵, wherein R⁵ is as defined above, such as H₂NOCH₂COOEt;
Me₃Si(CH₂)ₙONH₂, wherein n is as defined above, such as Me₃Si(CH₂)₂ONH₂;
AlkONH₂, wherein Alk is a C₅-C₁₀ linear, branched or cyclic alkyl group, such as C₈H₁₇ONH₂;
Ph₃CONH₂, wherein Ph stands for phenyl; or
R⁷C₆H₄CH₂CHR⁸ONH₂, wherein R⁷ represents a hydrogen atom or a OMe, OH, C₁₋₄ alkyl group, a COOH or COOR⁵ group, R⁵ being as defined above, and R⁸ represents a hydrogen atom or, taken together with R⁷ represents a CH₂ group, such as O—(2-phenylethyl)hydroxylamine or 2-(aminooxy)indane.

Furthermore, the compounds of formula (I) may be in their protonated or unprotonated form. By “protonated form” we mean here the addition of a proton to the —NH₂ group to form a —NH₃⁺ unit. Compounds of this type include in particular hydrochloride or hydrobromide derivatives of the compounds according to formula (I). Protonation and deprotonation is dependent on the pH of the medium, under highly acidic conditions for example compounds of formula (I) are expected to be in their protonated form.

It is also important to mention that, as a person skilled in the art can foresee, some of the derivatives of formula (I) can form lipid assemblies such as micelles or liposomes.

Furthermore, in all the above embodiments of the invention, the derivatives of formula (I) which are odorless, i.e. do not possess as such a significant odor, or are even essentially non-volatile represent particularly appreciated examples.

Amongst the above-mentioned dynamic mixtures many are also new, and therefore are also another object of the invention. Furthermore said new dynamic mixtures are also examples of said dynamic mixtures containing derivatives of formula (I) which are odorless, or a reduced volatility.

So another aspect of the present invention are the dynamic mixtures, for the controlled release of active ketones or aldehydes, obtainable by reversibly reacting, in a water-containing medium,
i) at least one derivative of formula

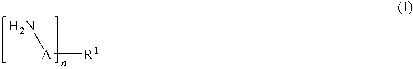

wherein:
A represents a functional group selected from the group consisting of the formulae (i) to (vi)

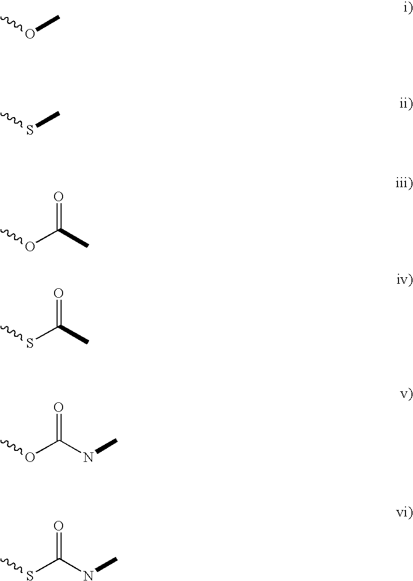

in which formulae the wavy line indicates the location of the bond between said NH₂ group and A, the bold lines indicate the location of the bond between said A and R¹; and
I) n is an integer varying from 1 to 4; and
   R¹ represents a mono-, di-, tri- or tetra-radical derived from a
      C₁ to C₁₈ linear, branched, cyclic or multicyclic hydrocarbon comprising one, two, or three functional groups selected from the group consisting of silyl, ketone, amide, ether, thioether or secondary or tertiary amines; said R¹ being optionally substituted by up to three groups selected from the group consisting of NR²₂, NHR², (NR²R³₂)X, OR³, SO₃M, R³ and COOR⁴, R² representing a C₁ to C₁₀ alkyl, acyl or polyethylene- or polypropylene-glycol group, a phenyl group or a C₆ to C₉ alkylaryl group, and said R³ representing a hydrogen atom or a R² group, M representing a hydrogen atom or an alkali metal ion, R⁴ representing a M group or a R³ group and X representing a halogen atom or a sulphate;
      C₁ to C₁₈ polyethylene- or polypropylene-glycol group; or
      C₅ to C₁₈ linear, branched, cyclic or multicyclic alkyl or alkenyl group; or
      a phenyl group, optionally substituted by up to three groups selected from the group consisting of NR²₂, NHR², (NR²R³₂)X, OR³, SO₃M, R³ and COOR⁴, R² representing a C₁ to C₁₀ alkyl, acyl or polyethylene- or polypropylene-glycol group, a phenyl group or a C₆ to C₉ alkylaryl group, and said R³ representing a hydrogen atom or a R² group, M representing a hydrogen atom or an alkali metal ion, R⁴ representing a M group or a R³ group and X representing a halogen atom or a sulphate; or
      a C₈ to C₂₀ linear, branched, cyclic or multicyclic hydrocarbon, optionally substituted by up to three groups selected from the group consisting of NR²₂, NHR², (NR²R³₂)X, OR³, SO₃M, R³ and COOR⁴, R² representing a C₁ to C₁₀ alkyl, acyl or polyethylene- or polypropylene-glycol group, a phenyl group or a C₆ to C₉ alkylaryl group, and said R³ representing a hydrogen atom or a R² group, M representing a hydrogen atom or an alkali metal ion, R⁴ representing a M group or a R³ group and X representing a halogen atom or a sulphate;
II) n is an integer varying from 2 to 5000; and R¹ represents a polyalkylene chain comprising between 2 and 5000 monomeric units;
   with
ii) at least one active aldehyde or ketone having a molecular weight comprised between 80 and 230 g/mol and being a perfuming, flavoring, insect repellent or attractant, bactericide or fungicide ingredient, in particular being selected from the group consisting of the C₅₋₂₀ perfuming aldehydes and the C₅₋₂₀ perfuming ketones.

According to a particular embodiment of said dynamic mixture, the compounds of formula (I) are those wherein
A represents a functional group selected from the group consisting of the formulae (i) or (ii) as defined above; and
I) n is an integer varying from 1 to 4; and
   R¹ represents a mono-, di-, tri- or tetra-radical derived from a
      C₃ to C₁₂ linear, branched, cyclic or multicyclic hydrocarbon comprising one, two, or three functional groups selected from the group consisting of silyl, ketone, amide, ether, thioether or secondary or tertiary amines; said hydrocarbon being optionally substituted by up to three groups selected from the group consisting of OR³, SO₃M and COOR⁴, M representing a hydrogen atom or an alkali metal ion, R³ representing a hydrogen atom or a C₁ to C₆ alkyl, acyl or polyethylene- or polypropylene-glycol group, a phenyl group or a C₆ to C₉ alkylaryl group, and R⁴ representing a M group or a hydrogen atom;
      C₃ to C₁₀ polyethylene- or polypropylene-glycol group; or
      C₅ to C₁₂ linear, branched, cyclic or multicyclic alkyl or alkenyl group; or
      a phenyl group optionally substituted by up to three groups selected from the group consisting of OR³, SO₃M and COOR⁴, M representing a hydrogen atom or an alkali metal ion, R³ representing a hydrogen atom or a C₁ to C₆ alkyl, acyl or polyethylene- or polypropylene-glycol group, a phenyl group or a C₆ to C₉ alkylaryl group, and R⁴ representing a M group or a hydrogen atom; or
      a C₈ to C₂₀ linear, branched, cyclic or multicyclic hydrocarbon comprising one to three C₆ aromatic moieties, optionally substituted by up to three groups selected from the group consisting of OR³, SO₃M and COOR⁴, M representing a hydrogen atom or an alkali metal ion, R³ representing a hydrogen atom or a C₁ to C₆ alkyl, acyl or polyethylene- or polypropylene-glycol group, a phenyl group or a C₆ to C₉ alkylaryl group, and R⁴ representing a M group or a hydrogen atom;
II) n is an integer varying from 2 to 5000; and R¹ represents a polyalkylene chain comprising between 2 and 5000 monomeric units.

The dynamic mixtures wherein A represents an oxygen atom are particularly appreciated.

More specifically, as non-limiting examples of O-substituted hydroxylamine or S-substituted thiohydroxylamine derivatives which are particularly useful in the invention's delivery systems, one may cite the following classes:
i) Ar(CO)ₐONH₂, Ar—NH—CO—ONH₂, Ar(CO)ₐSNH₂ or Ar—NH—CO—SNH₂, wherein a is 1 or 0 and Ar is a substituted or non-substituted C₆₋₉ phenyl group, such as phenyl or tolyl or C₆H₄COOH;
ii) Alk(CO)ₐONH₂, Alk-NH—CO—ONH₂, Alk(CO)ₐSNH₂ or Alk-NH—CO—SNH₂, wherein a is 1 or 0 and Alk is a C₅-C₁₈ linear, branched or cyclic alkyl group, optionally substituted by two OH groups, or a C₅-C₁₈ linear, branched or cyclic alkyl group, such as a C₅, C₆, C₈, C₁₂, C₁₆, C₁₈ group;
iii) (H₂NO(CO)ₐ)ₛ-Alk or (H₂NS(CO)ₐ)ₛ-Alk, wherein a and Alk are as defined above and s is 2, 3 or 4;
iv) H₂NO(CO)ₐ—(CH₂)ₚ-T, wherein a is defined as above, p is an integer varying from 1 to 12 and T is a COOM, COOR⁵, SO₃M or N(R⁵)₃X group, M represents a Na or K cation, X represents a Cl or sulphate anion and R⁵ represents a C₁-C₄ alkyl group;
v) H₂NO(COCH₂O)ₐ—(CH₂CH₂O)_{q}—U or H₂NO(COCH₂O)ₐ—(CH₂CH₂O)_{q}—(CH₂CO)ₐONH₂ wherein a is defined as above, q is an integer varying from 1 to 9 and U is a hydrogen atom or a CH₃ group;
vi) H₂NS(COCH₂O)ₐ—(CH₂CH₂O)_{q}—U or H₂NS(COCH₂O)ₐ—(CH₂CH₂O)_{q}—(CH₂CO)ₐSNH₂ wherein a is defined as above, q is an integer varying from 1 to 9 and U is a hydrogen atom or a CH₃ group;
vii) (H₂NO(CO)ₐ(CH₂)₂₋ₐ)ᵣ(R⁶)₂₋ᵣNCH₂CH₂N(R⁶)₂₋ᵣ(CH₂)₂₋ₐ(CO)ₐONH₂)ᵣ or (H₂NO(CO)ₐ(CH₂)₂₋ₐ)ₘ(R⁶)₃₋ₘN, wherein a is defined as above, r is 1 or 2, m is 1, 2 or 3 and R⁶ is a hydrogen atom or a C₁-C₄ alkyl group;
viii) Q((CH₂)ₐCOOR⁶)₃₋ₘ((CH₂)ₐ(CO)ₐONH₂)ₘ, wherein R⁶ is as defined above, m is 1, 2 or 3 and Q is N or COR^{E}, a being as defined above;
ix) Ph₃CONH₂, wherein Ph stands for phenyl;
x) R⁵₃Si(CH₂)ₙONH₂, wherein R⁵ and n are as defined above, or
xi) an O-substituted poly-hydroxylamine or an S-substituted poly-thiohydroxylamine derivative of poly(methyl methacrylate) and co-polymers thereof, of poly(methyl acrylate) and co-polymers thereof or of poly(4-vinylbenzoates) and co-polymers thereof.

In particular, there can be used the above-mentioned derivatives of O-substituted hydroxylamine which may be further exemplified by the following compounds:
H₂NO—(CH₂CH₂O)_{q}-Me or H₂NO—(CH₂CH₂O)_{q}—CH₂CH₂ONH₂, wherein q is an integer varying from 1 to 4, such as MeOCH₂CH₂ONH₂; H₂NOCH₂CH₂OCH₂CH₂OCH₂CH₂OMe or H₂NOCH₂CH₂OCH₂CH₂OCH₂CH₂ONH₂;
H₂NOCH₂COOR⁵, wherein R⁵ is as defined above, such as H₂NOCH₂COOEt;
Me₃Si(CH₂)ₙONH₂, wherein n is as defined above, such as Me₃Si(CH₂)₂ONH₂;
AlkONH₂, wherein Alk is a C₅-C₁₀ linear, branched or cyclic alkyl group, such as C₈H₁₇ONH₂;
Ph₃CONH₂, wherein Ph stands for phenyl; or
R⁷C₆H₄—CH₂CHR⁸ONH₂, wherein R⁷ represents a hydrogen atom or a OMe, OH, C₁₋₄ alkyl group, a COOH or COOR⁵ group, R⁵ being as defined above, and R⁸ represents a hydrogen atom or, taken together with R⁷ represents a CH₂ group, such as O-(2-phenylethyl)hydroxylamine or 2-(aminooxy)indane.

According to a particular embodiment of the invention, the invention's delivery systems and the use thereof are particularly appreciated when the derivative of formula (I) is a derivative comprising a polyethylene- or polypropylene-glycol group as defined above.

In all the aspects of the above-described invention the delivery systems may further comprise other amine derivatives known to generate dynamic mixtures, and in particular the hydrazine derivatives mentioned in WO2006/016248.

In all the aspects of the above-described invention active aldehydes or ketones, and in particular the perfuming ones, are mentioned. Said active ingredients are another important element of the dynamic mixture according to the present invention.

Said perfuming compounds comprise, preferably, 6 to 20 carbon atoms or even between 7 and 15 carbon atoms.

According to an embodiment of the invention, said perfuming aldehyde or ketone has a molecular weight comprised between 90 or 100 and 220 g/mol and can be advantageously selected from the group consisting of an enal, an enone, an aldehyde comprising the moiety CH₂CHO or CHMeCHO, an aryl aldehyde or ketone (wherein the functional group is directly bound to an aryl ring) and a cyclic or acyclic ketone (wherein the CO group is part or not of a cycle).

Furthermore, according to any of the embodiments mentioned above, said perfuming aldehyde or ketone is advantageously characterized by a vapor pressure above 2.0 Pa, as obtained by calculation using the software EPIwin v 3.10 (available at 2000 US Environmental Protection Agency). According to another embodiment, said vapor pressure is above 5.0, or even above 7.0 Pa.

As mentioned further above, all these embodiments apply also in the case of the active ingredient being a flavoring, insect repellent or attractant, bactericide or fungicide ingredient.

More specifically, as non-limiting examples of the perfuming compounds in the embodiments mentioned above, one may cite the following:
A) aldehydes of formula R″—CHO wherein R″ is a linear or α-branched alkyl group of C₆ to C₁₂, benzaldehyde, 1,3-benzodioxol-5-carboxaldehyde (heliotropine), 3-(1,3-benzodioxol-5-yl)-2-methylpropanal, 2,4-decadienal, 2-decenal, 4-decenal, 8-decenal, 9-decenal, 3-(6,6-dimethyl-bicyclo[3.1.1]hept-2-en-2-yl)propanal, 2,4-dimethyl-3-cyclohexene-1-carbaldehyde (Triplal®, origin: International Flavors & Fragrances, New York, USA), 3,5-dimethyl-3-cyclohexene-1-carbaldehyde, 1-(3,3-dimethyl-1-cyclohexyl)-1-ethanone, 5,9-dimethyl-4,8-decadienal, 2,6-dimethyl-5-heptenal (melonal), 3,7-dimethyl-2,6-octadienal (citral), 3,7-dimethyloctanal, 3,7-dimethyl-6-octenal (citronellal), (3,7-dimethyl-6-octenyl)acetaldehyde, 3-dodecenal, 4-dodecenal, 3-ethoxy-4-hydroxybenzaldehyde (ethyl vanillin), 4-ethyl benzaldehyde, 3-(2 and 4-ethylphenyl)-2,2-dimethylpropanal, 2-furancarbaldehyde (furfural), 2,4-heptadienal, 4-heptenal, 2-hexyl-3-phenyl-2-propenal (hexylcinnamic aldehyde), 2-hydroxybenzaldehyde, 7-hydroxy-3,7-dimethyloctanal (hydroxycitronellal), 4-hydroxy-3-methoxybenzaldehyde (vanillin), 4- and 3-(4-hydroxy-4-methylpentyl)-3-cyclohexene-1-carbaldehyde (Lyral®, origin: International Flavors and Fragrances, New York, USA), 4-isopropylbenzaldehyde (cuminaldehyde), 3-(4-isopropylphenyl)-2-methylpropanal, 2-(4-isopropylphenyl)propanal, 1,8-p-menthadien-7-al, (4R)-1-p-menthene-9-carbaldehyde (Liminal®, origin: Firmenich SA, Geneva, Switzerland), 2- and 4-methoxybenzaldehyde (anis aldehyde), 6-methoxy-2,6-dimethylheptanal (methoxymelonal), 8 (9)-methoxy-tricyclo[5.2.1.0.(2,6)]decane-3 (4)-carbaldehyde (Scentenal®, origin: Firmenich SA, Geneva, Switzerland), 4-methylbenzaldehyde, 2-(4-methylenecyclohexyl)propanal, 1-methyl-4-(4-methyl-3-pentenyl)-3-cyclohexen-1-carbaldehyde (Precyclemone® B, origin: International Flavors & Fragrances, New York, USA), 4-(4-methyl-3-pentenyl)-3-cyclohexene-1-carbaldehyde (Acropal®, origin: Givaudan-Roure SA., Vernier, Switzerland), (4-methylphenoxy)acetaldehyde, (4-methylphenyl)acetaldehyde, 3-methyl-5-phenylpentanal, 2-(1-methylpropyl)-1-cyclohexanone, 2,4-nonadienal, 2,6-nonadienal, 2-nonenal, 6-nonenal, 8-nonenal, 2-octenal, phenoxyacetaldehyde, phenylacetaldehyde, 3-phenylbutanal (Trifernal®, origin: Firmenich SA, Geneva, Switzerland), 3-phenylpropanal, 2-phenylpropanal (hydratropaldehyde), 3-phenyl-2-propenal (cinnamic aldehyde), 3-(4-tert-butylphenyl)-2-methylpropanal (Lilial®, origin: Givaudan-Roure SA, Vernier, Switzerland), 3-(4-tert-butylphenyl)propanal (Bourgeonal®, origin: Quest International, Naarden, Netherlands), tricyclo[5.2.1.0(2,6)]decane-4-carbaldehyde, exo-tricyclo[5.2.1.0(2,6)]decane-8exo-carbaldehyde (Vertral®, origin: Symrise, Holzminden, Germany), 2,6,6-trimethyl-bicyclo[3.1.1]heptane-3-carbaldehyde (formyl pinane), 2,4,6- and 3,5,6-trimethyl-3-cyclohexene-1-carbaldehyde, 2,2,3-trimethyl-3-cyclopentene-1-acetaldehyde (campholenic aldehyde), 2,6,10-trimethyl-2,6,9,11-dodecatetraenal, 2,5,6-trimethyl-4-heptenal, 3,5,5-trimethylhexanal, 2,6,10-trimethyl-9-undecenal, 2-undecenal, 10-undecenal or 9-undecenal and their mixtures such as Intreleven aldehyde (origin: International Flavors & Fragrances, New York, USA), and
B) C₆₋₁₁ ketones of formula R′—(CO)—R″ wherein R′ and R″ are linear alkyl groups, damascenones and damascones, ionones and methyl ionones (such as Iralia® Total, origin: Firmenich SA, Geneva, Switzerland), irones, macrocyclic ketones such as, for example, cyclopentadecanone (Exaltone®) or 3-methyl-4-cyclopentadecen-1-one and 3-methyl-5-cyclopentadecen-1-one (Delta Muscenone) or 3-methyl-1-cyclopentadecanone (Muscone) all from Firmenich SA, Geneva, Switzerland, 1-(2-aminophenyl)-1-ethanone, 1-(5,5-dimethyl-1-cyclohexen-1-yl)-4-penten-1-one (Neobutenone®, origin: Firmenich SA, Geneva, Switzerland), 1-(3,3-dimethyl-1-cyclohexyl)-1-ethanone, 2,5-dimethyl-2-octene-6-one, 4,7-dimethyl-6-octene-3-one, (3,7-dimethyl-6-octenyloxy)acetaldehyde, 1-(2,4-dimethylphenyl)-1-ethanone, 4-(1,1-dimethylpropyl)-1-cyclohexanone (Orivone®, origin: International Flavors & Fragrances, New York, USA), 2,4-di-tert-butyl-1-cyclohexanone, ethyl 4-oxopentanoate, 1-(4-ethylphenyl)-1-ethanone, 2-hexyl-1-cyclopentanone, 2-hydroxy-3-methyl-2-cyclopenten-1-one, 4-(4-hydroxy-1-phenyl)-2-butanone (raspberry ketone), 1-(2- and 4-hydroxyphenyl)-1-ethanone, 4-isopropyl-2-cyclohexen-1-one, 1-(4-isopropyl-1-phenyl)-1-ethanone, 1(6),8-p-menthadien-2-one (carvone), 4(8)-p-menthen-3-one, 1-(1-p-menthen-2-yl)-1-propanone, menthone, (1R,4R)-8-mercapto-3-p-menthanone, 1-(4-methoxyphenyl)-1-ethanone, 7-methyl-2H,4H-1,5-benzodioxepin-3-one (Calone®, origin: C.A.L. SA, Grasse, France), 5-methyl-3-heptanone, 6-methyl-5-hepten-2-one, methyl 3-oxo-2-pentyl-1-cyclopentaneacetate (Hedione®, origin: Firmenich SA, Geneva, Switzerland), 1-(4-methylphenyl)-1-ethanone (4-methylacetophenone), 5-methyl-exo-tricyclo[6.2.1.0(2,7)]undecan-4-one, 3-methyl-4-(1,2,2-trimethylpropyl)-4-penten-2-one, 2-naphthalenyl-1-ethanone, 1-(octahydro-2,3,8,8-tetrame-2-naphthalenyl)-1-ethanone (isomeric mixture, Iso E Super®, origin: International Flavors & Fragrances, New York, USA), 3,4,5,6,6-pentamethyl-3-hepten-2-one, 2-pentyl-1-cyclopentanone (Delphone, origin: Firmenich SA, Geneva, Switzerland), 4-phenyl-2-butanone (benzylacetone), 1-phenyl-1-ethanone (acetophenone), 2- and 4-tert-butyl-1-cyclohexanone, 1-(4-tert-butylphenyl)-1-ethanone), 2,4,4,7-tetramethyl-6-octen-3-one, 1,7,7-trimethyl-bicyclo[2.2.1]heptan-2-one (camphor), 2,6,6-trimethyl-1-cycloheptanone, 2,6,6-trimethyl-2-cyclohexene-1,4-dione, 4-(2,6,6-trimethyl-2-cyclohexen-1-yl)-2-butanone (dihydroionone), 1-(2,4,4-trimethyl-2-cyclohexen-1-yl)-2-buten-1-one, 1-(3,5,6-trimethyl-3-cyclohexen-1-yl)-1-ethanone, 2,2,5-trimethyl-5-pentyl-1-cyclopentanone;
   wherein the underlined compounds represent, in an embodiment of the invention, particularly useful fragrance aldehydes or ketones.

Furthermore, some of the above-mentioned compounds may also be used as perfuming, flavoring, insect repellent or attractant, bactericide or fungicide ingredients.

The invention's dynamic mixture can be obtained by admixing together, in the presence of water, at least one compound of formula (I) and at least one perfuming compound. Furthermore, as it is very useful in the perfumery art to have compounded perfumery ingredients, so as to achieve more pleasant and natural scents, a dynamic mixture obtained by reacting together at least two, or even at least three, derivatives of formula (I) with at least one perfuming compound is particularly appreciated. Similarly, it is also particularly appreciated to obtain a dynamic mixture by reacting together at least one or two derivatives of formula (I) with at least two, or even at least three, perfuming compounds.

As mentioned above, the invention's dynamic mixture comprises several starting components that may react, in a reversible manner, between them to form addition products.

It is believed that the main components of the dynamic mixture are the free aldehyde and/or ketone, the derivatives of formula (I) and the resulting addition products (such as the corresponding R¹AN═CR₂ derivatives, CR₂ representing a residue of the active aldehyde or ketone, or also the corresponding “hemiaminal”). A specific example of such a mixture and equilibrium is presented in Scheme (I):
Scheme (I): Example of an equilibrium and the species present in a dynamic mixture obtained from one specific aldehyde and one specific RONH₂ derivative or from the corresponding O-alkyloxime derivative.

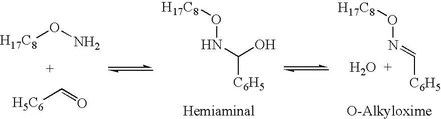

As a consequence of the fact that the reactions are reversible, a dynamic mixture can also be obtained by adding one or more R′AN═CR₂ compounds into water and let the mixture attain its equilibrium. However, it has to be pointed out that the time required to reach the equilibrium point can vary significantly depending on the fact that there is used, for instance, the derivative of formula (I) as starting material, as said time is believed to be dependent on various parameters such as solubilities or the basicity of the medium.

The preparation of the invention's dynamic mixture by the simple admixture of the perfuming compounds and of the derivative of formula (I) in the presence of water avoids the need of additional chemical steps such as the preparation of the corresponding R¹AN═CR₂.

Consequently, the R¹AN═CR₂ compounds, being precursors of the invention dynamic mixtures are also an object of the present invention, and in particular those of formula

R⁹ON═CR₂ (III)

wherein CR₂ represents a residue of the active aldehyde or ketone (O═CR₂) as above described, and R⁹ represents
a C₃-C₁₈ group of formula —(CH₂CH₂O)ₓH or —(CH₂CH₂O)_{w}CHR¹⁰₂, x representing an integer from 1 to 9, w representing an integer varying from 1 to 8, R¹⁰ representing, independently from each other, a hydrogen atom or a hydrocarbon group, optionally comprising one, two, or three functional groups selected from the group consisting of ketone, amide, ether, alcohol, or secondary or tertiary amines;
a group of formula —(CH₂CH₂O)_{w}ON═CR₂, CR₂ being defined as above and w representing an integer varying from 1 to 9;
—(CH₂CH₂O)_{w}C₆H₅, w being as defined above;
—CH₂CH₂SiMe₃; or
—C₆H₁₁;
   provided that cyclohexanone O—[2-(ethenyloxy)ethyl]oxime, 1-phenyl-ethanone O—[2-(ethenyloxy)ethyl]oxime, 1-phenyl-ethanone O-[2-[(tetrahydro-2H-pyran-2-yl)oxy]ethyl]oxime, 1-(4-methoxyphenyl)-ethanone O-[2-[(tetrahydro-2H-pyran-2-yl) oxy]ethyl]oxime, 4-phenyl-3-buten-2-one O-[2-[(4-methoxyphenyl)methoxy]ethyl]oxime, benzaldehyde O-[2-(2-cyclohexen-1-yloxy)ethyl]oxime, 2,3-dihydro-1H-inden-1-one O-[2-[(tetrahydro-2H-pyran-2-yl)oxy]ethyl]oxime, 1-(1,3-benzodioxol-5-yl)-ethanone O-[2-[(tetrahydro-2H-pyran-2-yl)oxy]ethyl]oxime, 1-(2-naphthalenyl)-ethanone O-[2-[(tetrahydro-2H-pyran-2-yl)oxy]ethyl]oxime, 2-naphthalenecarboxaldehyde O-[2-[(tetrahydro-2H-pyran-2-yl)oxy]ethyl]oxime, benzaldehyde O-[2-[(tetrahydro-2H-pyran-2-yl)oxy]ethyl]oxime, benzaldehyde O-(2-methoxyethyl)oxime, 4-methoxy-benzaldehyde O,O′-[1,2-ethanediylbis(oxy-2,1-ethanediyl)]dioxime, 2-hydroxy-benzaldehyde O,O′-[oxybis(2,1-ethanediyloxy-2,1-ethanediyl)]dioxime, 2-methoxy-benzaldehyde O,O′-[oxybis(2,1-ethanediyloxy-2,1-ethanediyl)]dioxime, 2-methoxy-benzaldehyde O,O′-(oxydi-2,1-ethanediyl)dioxime, 2-undecanone O-(26-hydroxy-3,6,9,12,15,18,21,24-octaoxahexacos-1-yl)oxime, 2-undecanone O-(17-hydroxy-3,6,9,12,15-pentaoxaheptadec-1-yl)oxime, 2-undecanone O-[2-[2-(2-hydroxyethoxy)ethoxy]ethyl]oxime, cyclohexanone O-[2-(oxiranyloxy)ethyl]oxime, benzaldehyde O-[2-(oxiranyloxy)ethyl]oxime and 2-undecanone O-[2-(2-hydroxyethoxy)ethyl]oxime are excluded.

According to a particular embodiment said compound of formula (III) is a compound wherein R⁹ represents
a C₃-C₁₈ group of formula —(CH₂CH₂O)ₓH or —(CH₂CH₂O)_{w}CHR¹⁰₂, x representing an integer varying from 1 to 9, w representing an integer from 1 to 8, R¹⁰ representing, independently from each other, a hydrogen atom or a C₁-C₆ hydrocarbon group;
a group of formula —(CH₂CH₂O)_{w}ON═CR₂, CR₂ being defined as above w representing an integer varying from 1 to 9;
—(CH₂CH₂O)_{w}C₆H₅, w being as defined above.

Now, due to its nature, the invention's dynamic mixture circumvents the problem of product instability observed with prior art precursors, by the fact that a dynamic equilibrium is spontaneously set up between these compounds. The equilibrium is stable during product storage as long as the consumer product parameters (such as concentration, temperature, pH or humidity, the presence of surfactant etc.) are kept constant. At a given set of parameters, the time required to reach the equilibrium state mainly depends on the kinetic rate constant of the slowest step involved in the formation of the products of the equilibrium.

The invention's dynamic mixture is furthermore able to stabilize active aldehydes and ketones, against degradation, in aqueous media by reversibly forming an addition product between a compound of formula (I) and the active aldehyde or ketone and thus reversibly protects the carbonyl function as an oxime or thioxime function. The spontaneous reversible formation of a high amount of oximes or thioximes in the dynamic mixture is thus expected to stabilize the carbonyl functionality of the active aldehyde or ketone to a large extent.

As mentioned above, the dynamic mixture of the invention comprises various components. It is believed that, once the dynamic mixture is deposited on a surface, the free perfuming aldehydes or ketones start to evaporate, diffusing in the surrounding environment their typical scent. Said evaporation perturbs the chemical equilibrium and the various addition products start to decompose so as to restore the equilibrium. The consequence of such re-equilibration is the regeneration of free perfuming aldehydes or ketones, thus maintaining their concentration relatively constant over time and avoiding a too rapid evaporation.

Now, it has been observed that the various physical or thermodynamic properties of the dynamic mixture, e.g. its deposition on a surface or the amount of addition products formed, can be influenced by the chemical nature of the perfuming compounds or of the derivatives of formula (I). Another way to influence the above-mentioned properties is to modify the molar ratio between said perfuming compounds and the derivatives of formula (I). For instance, the lower the molar ratio between perfuming compounds and derivatives of formula (I), the longer takes the evaporation of all the perfuming compounds. The presence of other ingredients (such as surfactants, emulsifiers, gelators or others) typically used in the final consumer product formulation may also influence the above-mentioned properties.

Therefore, by varying the chemical structure of the mixture's constituents and their ratio, it is possible to fine-tune the release properties of the invention's dynamic mixture, so as to adapt its behavior to the specific requirement of the targeted consumer product.

According to the final application, a broad range for the speed of evaporation of the perfuming compound may be desirable.

The ratio between the total molar amount of perfuming aldehyde and/or ketone and the total molar amount of the compound of formula (I) can be comprised between 1:2 and 50:1, preferably between 1:1 and 10:1.

The amount of free active aldehyde or ketone present in the equilibrated dynamic mixture is comprised between 1 and 97%, preferably between 5 and 95% or even more preferably between 25 and 90%.

Another advantage of the invention resides in the fact that it is possible to fine-tune the thermodynamic behavior of the dynamic mixture by selecting the nature of A and R¹ groups. It is therefore conceivable to design dynamic mixtures comprising, for instance, a derivative of formula (I) which allows a fast release of a specific active aldehyde (which will be perceivable at the beginning of the consumer use only) and a second derivative of formula (I) which allows a release of the same specific aldehyde, or of another, a very slow release (which will be perceivable even after an important delay from the direct consumer use).

Moreover, another object of the present invention concerns also a composition comprising the invention's dynamic mixture. This concerns also in particular a perfuming composition comprising:
i) as perfuming ingredient, a dynamic mixture as defined above;
ii) at least one ingredient selected from the group consisting of a perfumery carrier and a perfumery base; and
iii) optionally at least one perfumery adjuvant.

Preferably, in said perfuming composition the perfumery carrier, perfumery base and perfumery adjuvant have a total molar amount of aldehydes or ketones which is equal to or higher than the molar amount of derivatives of formula (I) of the dynamic mixture.

By “perfumery carrier” we mean here a material which is practically neutral from a perfumery point of view, i.e. that does not significantly alter the organoleptic properties of perfuming ingredients. Said carrier may be a liquid. As liquid carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the ones most commonly used.

By “perfumery base” we mean here a composition comprising at least one perfuming co-ingredient.

Said perfuming co-ingredient is not an aldehyde or ketone as defined above for the dynamic mixture. Moreover, by “perfuming co-ingredient” it is meant here a compound, which is used in perfuming preparation or composition to impart a hedonic effect. In other words such a co-ingredient, to be considered as being a perfuming one, must be recognized by a person skilled in the art as being able to impart or modify in a positive or pleasant way the odor of a composition, and not just as having an odor.

The nature and type of the perfuming co-ingredients present in the base do not warrant a more detailed description here, which in any case would not be exhaustive, the skilled person being able to select them on the basis of its general knowledge and according to intended use or application and the desired organoleptic effect. In general terms, these perfuming co-ingredients belong to chemical classes as varied as alcohols, esters, ethers, acetates, nitriles, terpene hydrocarbons, nitrogenous or sulphurous heterocyclic compounds and essential oils, and said perfuming co-ingredients can be of natural or synthetic origin. A further class of perfuming co-ingredients can be the aldehydes or ketones which do not react with the O-substituted hydroxylamine or S-substituted thiohydroxylamine derivative present in the dynamic mixture.

Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said co-ingredients may also be compounds known to release in a controlled manner various types of perfuming compounds.

For the compositions which comprise both a perfumery carrier and a perfumery base, other suitable perfumery carriers than those previously specified, can also be ethanol, water/ethanol mixtures, limonene or other terpenes, isoparaffins such as those known under the trademark Isopar (origin: Exxon Chemical) or glycol ethers and glycol ether esters such as those known under the trademark Dowanol® (origin: Dow Chemical Company).

By “perfumery adjuvant” we mean here an ingredient capable of imparting additional added benefit such as a color, a particular light resistance, chemical stability and others. A detailed description of the nature and type of adjuvant commonly used in perfuming bases cannot be exhaustive, but it has to be mentioned that said ingredients are well known to a person skilled in the art.

An invention's composition consisting of an invention's dynamic mixture and at least one perfumery carrier represents a particular embodiment of the invention as well as a perfuming composition comprising an invention's dynamic mixture, at least one perfumery carrier, at least one perfumery base, and optionally at least one perfumery adjuvant.

As anticipated above, the invention's dynamic mixtures or compositions can be advantageously used for bringing a benefit to consumer products, such as its perfuming. Indeed, said mixture possesses several other properties that make it particularly suitable for this purpose. Consequently, a consumer article comprising the invention's dynamic mixture is also an object of the present invention.

Indeed, and for example, another advantage of the invention's mixture is an improved deposition on a surface of the perfuming aldehydes or ketones compared to those of the pure aldehydes or ketones as such.

All the above-mentioned properties, i.e. improved substantivity, prolonged time of evaporation, improved stability over aggressive agents, and improved deposition, are very important for a perfuming composition. Indeed, when said compositions are intended for use in fine perfumery, the invention's mixture may allow the creation of new perfuming effects which are otherwise difficult to be achieved, such as a fresh green note being present over several hours. In the case of perfuming compositions intended for the functional perfumery, the above-mentioned properties are also very important. For example, perfuming ingredients present as such in washing compositions which have generally little staying-power on a surface are consequently often eliminated, for example in the rinsing water or upon drying of said surface. This problem can be solved by using the invention's dynamic mixture, which possesses an improved stability over storage and substantivity on surfaces, such as textiles or hair.

Therefore, the mixtures according to the invention, owing to a lower and more uniform evaporation per unit of time, resulting in a controlled release of odoriferous molecules, can be incorporated in any application requiring the effect of prolonged liberation of an odoriferous component as defined hereinabove and furthermore can impart a fragrance and a freshness to a treated surface which will last well beyond the rinsing and/or drying processes. Suitable surfaces are, in particular, textiles, hard surfaces, hair and skin.

Consequently, the invention concerns also in particular consumer article in the form of a perfumed article comprising:

i) as perfuming ingredient, a dynamic mixture as defined above; and
ii) a liquid consumer product base;
is also an object of the present invention.

Preferably, in perfumed articles the liquid consumer product base has a total molar amount of aldehydes and/or ketones which is equal to or higher than the molar amount of derivatives of formula (I) of the dynamic mixture.

For the sake of clarity, it has to be mentioned that, by “liquid consumer product base” we mean here a consumer product which is compatible with a perfume or perfuming ingredients and which is not a solid, e.g. a more or less viscous solution, a suspension, an emulsion, a gel or a cream. In other words, a perfumed article according to the invention comprises the functional formulation, as well as optionally additional benefit agents, corresponding to a consumer product, e.g. a conditioner, a softener or an air freshener, and an olfactivly effective amount of an invention's dynamic mixture.

It is also understood that said “liquid consumer product base” contains at least 10% w/w, or even 30% w/w, of water. More preferably, said base comprises at least 50% w/w, or even 70%, water optionally containing up to 30% of a surfactant. According to a particular embodiment of the invention, the base may have a pH comprised between 2 and 6.

The nature and type of the constituents of the liquid consumer product base do not warrant a more detailed description here, which in any case would not be exhaustive, the skilled person being able to select them on the basis of its general knowledge and according to the nature and the desired effect of said article.

Suitable consumer products comprise liquid detergents and fabric softeners as well as all the other articles common in perfumery, namely perfumes, colognes or after-shave lotions, perfumed liquid soaps, shower or bath mousses, oils or gels, hygiene products or hair care products such as shampoos, body-care products, liquid based deodorants or antiperspirants, air fresheners comprising a liquid perfuming ingredient and also cosmetic preparations. As detergents are intended applications such as detergent compositions or cleaning products for washing up or for cleaning various surfaces, e.g. intended for textile, dish or hard-surface treatment, whether they are intended for domestic or industrial use. Other perfumed articles are fabric refreshers, ironing waters, papers, wipes or bleaches.

Preferred consumer products are perfumes, air fresheners, cosmetic preparations, softener bases or hair care products.

According to an embodiment of the invention, it is also possible to have a perfumed article comprising:
i) a derivative of formula (I), as above described, and/or at least one O-alkyloxime obtainable from a derivative of formula (I) and an active aldehyde or ketone as above defined, and
   a perfume or perfuming composition containing at least one perfuming aldehyde or ketone having a molecular weight comprised between 80 and 230 g/mol;
   or
   at least one O-alkyloxime obtainable from a derivative of formula (I) and an active aldehyde or ketone as above defined;
      and
ii) a solid consumer product base intended to be used in the presence of water.

In such a case, the invention's dynamic mixture will be formed once the consumer article is used by the consumer, since water will be present. Examples of such solid consumer product bases intended to be used in the presence of water include powder detergents or “ready to use” powdered air fresheners. In particular, the O-alkyloxime cited above can be one of formula (III).

For the sake of clarity, it has to be mentioned that, by “O-alkyloxime obtainable from a derivative of formula (I) and an active aldehyde or ketone” we mean here a compound, as such, of formula R₁ON═CR₂ wherein R¹ and CR₂ are as defined above.

Typical examples of fabric detergents or softener compositions into which the compounds of the invention can be incorporated are described in Ullman's Encyclopedia of Industrial Chemistry, vol. A8, pages 315-448 (1987) and vol. A25, pages 747-817 (1994); Flick, Advanced Cleaning Product Formulations, Noye Publication, Park Ridge, N.J. (1989); Showell, in Surfactant Science Series, vol. 71: Powdered Detergents, Marcel Dekker, N.Y. (1988); Proceedings of the World Conference on Detergents (4th, 1998, Montreux, Switzerland), AOCS print.

Some of the above-mentioned articles may represent an aggressive medium for the invention's compounds, so that it may be necessary to protect the latter from premature decomposition, for example by encapsulation.

The proportions in which the dynamic mixture according to the invention can be incorporated into the various aforementioned articles or compositions vary within a wide range of values. These values are dependent on the nature of the article or product to be perfumed and on the desired olfactory effect as well as the nature of the co-ingredients in a given composition when the dynamic mixtures according to the invention are mixed with perfuming co-ingredients, solvents or additives commonly used in the art.

For example, typical concentrations are in the order of 0.1% to 30% by weight, or even more, of the invention's dynamic mixture based on the weight of the composition into which they are incorporated. Concentrations lower than these, such as in the order of 0.01% to 5% by weight, can be used when these dynamic mixtures are applied directly in the perfuming of the various consumer products mentioned hereinabove.

Another object of the present invention relates to a method for the perfuming of a surface characterized in that said surface is treated in the presence of a dynamic mixture as defined above. Suitable surfaces are, in particular, textiles, hard surfaces, hair and skin.

Moreover, an additional aspect of the present invention is a method for prolonging the perfuming effect of a perfuming aldehyde or ketone, as defined above, characterized in that there is added at least one derivative of formula (I), as defined above, to a perfuming composition containing at least one perfuming aldehyde or ketone, as defined above, and water. In other words, it concerns the use of a derivative of formula (I), as defined above, as additive to prolong the perfuming effect of a perfuming compositions containing at least one perfuming compound as defined above and water.

### EXAMPLES

The invention will now be described in further detail by way of the following examples, wherein the abbreviations have the usual meaning in the art, the temperatures are indicated in degrees centigrade (° C.). If not stated otherwise, the NMR spectral data were recorded on a Bruker AMX 400 spectrometer in DMSO-d₆ at 400 MHz for ¹H and at 100.6 MHz for ¹³C, the chemical displacements δ are indicated in ppm with respect to TMS as the standard, the coupling constants J are expressed in Hz. Commercially available reagents and solvents were used without further purification if not stated otherwise. Reactions were carried out in standard glassware under N₂.

Although specific conformations or configurations are indicated for some of the compounds, this is not meant to limit the use of these compounds to the isomers described. According to the invention, all possible conformation or configuration isomers are expected to have a similar effect.

The following O-substituted hydroxylamine or S-substituted thiohydroxylamine derivatives according to formula (I) can be obtained from commercial sources (some of which are sold as their corresponding hydrochloride salts): O-benzylhydroxylamine hydrochloride (origin: TCI), O-phenylhydroxylamine hydrochloride (origin: Fluka), O-(2-phenoxyethyl)hydroxylamine hydrochloride (origin: Interchim), Carboxymethoxylamine hemihydrochloride (origin: TCI), O-(2-trimethylsilylethyl)hydroxylamine hydrochloride (origin: TCI), O-(triphenylmethyl)hydroxylamine (origin: Fluka) and triphenyl-methanesulfenamide (origin: Aldrich).

Non commercial derivatives of formula (I) were prepared as follows:

### Preparation of O-octylhydroxylamine

### a) Synthesis of 2-(octyloxy)-1H-isoindole-1,3(2H)-dione

A solution of 2-hydroxy-1,3-isoindoledione (5.76 g, 35.2 mmol), octyl 4-methylbenzenesulfonate (10.00 g, 35.2 mmol) and 1,8-diazabicyclo[5.4.0]undec-7-ene (DBU, 5.35 g, 35.2 mmol) in DMF (300 ml) was heated at 80° C. for 17 h. The color of the reaction mixture turned from dark brown to clear yellow. The reaction mixture was concentrated, taken up in ethyl acetate, washed with a saturated solution of NaHCO₃ (3×) until the organic phase became colorless. Drying (Na₂SO₄) and concentrating gave 9.40 g (98%) of a yellow oil that slowly crystallizes.

¹H-NMR (CDCl₃): 7.88-7.81 (m, 2H); 7.79-7.71 (m, 2H); 4.20 (t, J=6.9, 2H); 1.85-1.74 (m, 2H); 1.54-1.42 (m, 2H); 1.41-1.20 (m, 8H); 0.88 (t, J=6.9, 3H).

¹³C-NMR (CDCl₃): 163.69 (s); 134.42 (d); 129.02 (s); 123.48 (d); 78.66 (t); 31.78 (t); 29.29 (t); 29.16 (t); 28.17 (t); 25.56 (t); 22.65 (t); 14.09 (q).

### b) Synthesis of O-octylhydroxylamine

Hydrazine hydrate (51% in water, 1.36 ml=1.39 g, 21.8 mmol) was added to a solution of 2-(octyloxy)-1H-isoindole-1,3(2H)-dione (6.00 g, 21.8 mmol) in methanol (150 ml). The reaction mixture turned orange and the formation of a precipitate was observed. After stirring at room temperature for 4 h, the reaction mixture was filtered. Concentration of the filtrate and re-filtration (2×) gave a total of 3.37 g (quant.) of a yellow oil.

¹H-NMR (CDCl₃): 5.34 (s br., 2H); 3.65 (t, J=6.7, 2H); 1.57 (quint., J=6.9, 2H); 1.41-1.18 (m, 10H); 0.88 (t, J=6.7, 3H).

¹³C-NMR (CDCl₃): 76.26 (t); 31.83 (t); 29.49 (t); 29.26 (t); 28.43 (t); 26.04 (t); 22.66 (t); 14.10 (q).

### Preparation of O-(cyclohexyl)hydroxylamine

### a) Synthesis of 2-(cyclohexyloxy)-1H-isoindole-1,3(2H)-dione

A solution of 2-hydroxy-1,3-isoindoledione (13.00 g, 79.7 mmol), cyclohexyl 4-methylbenzenesulfonate (20.00 g, 78.6 mmol) and DBU (11.90 g, 79.7 mmol) in DMF (200 ml) was heated at 80° C. for 17 h. After cooling to room temperature, the reaction mixture was concentrated, taken up in ethyl acetate and washed with a saturated solution of NaHCO₃ (3×). Drying (Na₂SO₄) and concentrating gave 5.10 g (26%) of a yellow oil.

¹H-NMR (CDCl₃): 7.84 (dd, J=3.1, 5.6, 2H); 7.75 (dd, J=3.1, 5.6, 2H); 4.24 (m, 1 H); 2.00-1.20 (m, 8H).

¹³C-NMR (CDCl₃): 164.34 (s); 134.39 (d); 129.01 (s); 123.43 (d); 85.68 (d); 30.80 (t); 25.30 (t); 23.71 (t).

### b) Synthesis of O-(cyclohexyl)hydroxylamine

Hydrazine hydrate (51% in water, 0.6 ml, 9.8 mmol) was added to a solution of 2-(cyclohexyloxy)-1H-isoindole-1,3(2H)-dione (2.30 g, 9.4 mmol) in methanol (30 ml). After stirring at room temperature for 14 h, the reaction mixture was concentrated and taken up in diethylether. A white solid was filtrated off and washed with ether before an aqueous solution of HCl (0.1 M, 20 ml) was added to the ether phase. After phase separation, the pH of the aqueous phase was adjusted to 10 by addition of an aqueous solution of NaOH (10%). After addition of diethylether (100 ml) and extraction, the organic layer was dried and concentrated to give 0.32 g (30%) of a yellow oil.

¹H-NMR (CDCl₃): 5.26 (br. s, 2H); 3.45-3.51 (m, 1H); 1.92-1.97 (m, 2H); 1.70-1.76 (m, 2H); 1.51-1.56 (m, 1H); 1.19-1.30 (m, 5H).

¹³C-NMR (CDCl₃): 81.86 (d); 31.02 (t); 25.87 (t); 23.93 (t).

### Preparation of 2-(aminooxy)indane

### a) Synthesis of 2-(2,3-dihydro-1H-inden-2-yloxy)-1H-isoindole-1,3(2H)-dione

A solution of 2-hydroxy-1,3-isoindoledione (5.00 g, 30.7 mmol), 2,3-dihydro-1H-inden-2-yl 4-methylbenzenesulfonate (8.83 g, 30.6 mmol) and DBU (4.66 g, 30.6 mmol) in DMF (290 ml) was heated at 80° C. for 23 h. The reaction mixture was concentrated, taken up in ethyl acetate and washed with a saturated solution of NaHCO₃ (3×). Drying (Na₂SO₄) and concentrating gave 4.99 g (58%) of a solid.

¹H-NMR (CDCl₃): 7.84 (dd, J=5.1, 3.1, 2H); 7.75 (dd, J=5.6, 3.1, 2H); 7.28-7.22 (m, 2H); 7.22-7.16 (m, 2H); 5.25 (m, 1H); 3.38 (AB, J=16.9, 3.1, 2H); 3.28 (AB, J=16.9, 6.1, 2H).

¹³C-NMR (CDCl₃): 164.21 (s); 139.90 (s); 134.53 (d); 128.93 (s); 126.91 (d); 124.73 (d); 123.57 (d); 88.64 (d); 38.54 (t).

### b) Synthesis of 2-(aminooxy)indane

Hydrazine hydrate (51% in water, 1.65 ml=1.69 g, 26.5 mmol) was added to a suspension of 2-(2,3-dihydro-1H-inden-2-yloxy)-1H-isoindole-1,3(2H)-dione (4.00 g, 14.3 mmol) in methanol (100 ml). The reaction mixture turned orange and the formation of a precipitate was observed. After stirring at room temperature for 6 h, the reaction mixture was filtered. Concentration of the filtrate and re-filtration gave a total of 2.11 g (53%) of a yellow oil.

¹H-NMR (CDCl₃): 7.26-7.19 (m, 2H); 7.19-7.12 (m. 2H); 5.33 (s br., 2H); 4.61-4.54 (m, 1H); 3.14 (AB, J=16.9, 5.6, 2H); 3.06 (AB, J=16.4, 3.1, 2H).

¹³C-NMR (CDCl₃): 141.04 (s); 126.51 (d); 124.78 (d); 84.44 (d); 38.36 (t).

### Preparation of O-(2-phenylethyl)hydroxylamine hydrochloride

### a) Synthesis of 2-(2-phenylethyloxy)-1H-isoindole-1,3(2H)-dione

A solution of 2-hydroxy-1,3-isoindoledione (12.90 g, 79.1 mmol), 2-phenylethyl 4-methylbenzenesulfonate (20.00 g, 72.4 mmol) and DBU (10.9 ml, 73.0 mmol) in DMF (200 ml) was heated at 80° C. for 17 h. The reaction mixture was concentrated, taken up in ethyl acetate and washed with a saturated solution of NaHCO₃ (3×). Drying (Na₂SO₄) and concentrating gave 19.40 g (99%) of a yellow solid.

¹H-NMR (CDCl₃): 7.82 (dd, J=3.1, 5.6, 2H); 7.73 (dd, J=3.1, 5.6, 2H); 7.29 (d, J=4.1, 4H); 7.24-7.17 (m, 1H); 4.43 (t, J=7.2, 2H); 3.14 (t, J=7.2, 2H).

¹³C-NMR (CDCl₃): 163.57 (s); 136.77 (s); 134.48 (d); 128.90 (d); 128.84 (d); 128.55 (d); 126.62 (d); 123.49 (d); 78.50 (t); 34.62 (t).

### b) Synthesis of O-(2-phenylethyl)hydroxylamine hydrochloride

Hydrazine hydrate (51% in water, 0.50 ml, 8.2 mmol) was added to a suspension of 2-(2-phenylethyloxy)-1H-isoindole-1,3(2H)-dione (2.30 g, 7.5 mmol) in methanol (10 ml). The reaction mixture turned orange and the formation of a precipitate was observed. After stirring at room temperature for 14 h, the reaction mixture was concentrated and taken up in diethylether. A white solid was filtrated off and washed with ether (3×). The organic phases were concentrated, taken up with diethylether previously saturated with HCl and re-concentrated give 0.75 g (58%) of an orange salt.

¹H-NMR: 11.12 (br. s); 7.20-7.34 (m, 5H); 4.26 (t, J=6.7, 2H); 2.94 (t, J=6.7, 2 H).

¹³C-NMR: 137.31 (s); 128.71 (d); 128.27 (d); 126.34 (d); 74.29 (t); 33.25 (t).

### Preparation of O-(2-methoxyethyl)hydroxylamine hydrochloride

### a) Synthesis of 2-(2-methoxyethoxy)-1H-isoindole-1,3(2H)-dione

A solution of 2-hydroxy-1,3-isoindoledione (10.00 g, 61.3 mmol), 2-methoxyethyl 4-methylbenzenesulfonate (14.10 g, 61.2 mmol) and DBU (9.30 g, 61.1 mmol) in DMF (500 ml) was heated at 80° C. for 17 h. The color of the reaction mixture turned from dark brown to clear yellow. The reaction mixture was concentrated, taken up in ethyl acetate, washed with a sat. solution of NaHCO₃ (3×) until the organic phase became colorless. Drying (Na₂SO₄) and concentrating gave 11.79 g (87%) of a white solid.

¹H-NMR (CDCl₃): 7.85 (dd, J=5.6, 3.1, 2H); 7.76 (dd, J=5.6, 3.1, 2H); 4.40-4.35 (m, 2H); 3.79-3.74 (m, 2H); 3.39 (s, 3H).

¹³C-NMR (CDCl₃): 163.45 (s); 134.45 (d); 128.99 (s); 123.52 (d); 77.07 (t); 70.45 (t); 59.12 (q).

### b) Synthesis of O-(2-methoxyethyl)hydroxylamine hydrochloride

Hydrazine hydrate (51% in water, 4.66 ml=4.68 g, 76.3 mmol) was added to a solution of 2-(2-methoxyethoxy)-1H-isoindole-1,3(2H)-dione (16.3 g, 73.6 mmol) in methanol (250 ml). After heating at 60° C. for 2.5 h and cooling to room temperature, the reaction mixture was filtered and concentrated to dryness. The residue treated with ether, filtered and the filtrate concentrated (2×) to give a slightly yellow oil. The oil was dissolved in ethanol (50 ml) and HCl (37%, 5 ml) was added. After stirring for 5 min the solvent was evaporated. Ether (50 ml) was added and evaporated (3×) to give a solid, which was washed with ether (20 ml, 2×) to give 4.00 g (43%) of an oil.

¹H-NMR: 11.19 (s br., 3H); 4.17 (t, J=4.4, 2H); 3.56 (t, J=4.5, 2H), 3.27 (s, 3H).

¹³C-NMR: 73.11 (d); 68.92 (d); 57.95 (t).

### Preparation of O-[2-[2-(2-methoxyethoxy)ethoxy]ethyl]hydroxylamine

### a) Synthesis of 2-[2-(2-methoxyethoxy)ethoxy]ethyl 4-methylbenzenesulfonate

4-Methyl-1-benzenesulfonyl chloride (70.0 g, 365.0 mmol) was slowly added to a solution of 2-[2-(2-methoxyethoxy)ethoxy]ethanol (50.0 g, 304.5 mmol) and triethylamine (37.0 g, 365 mmol) in DMF (200 ml) at 0° C. After stirring at room temperature for 1 h, the reaction mixture was poured onto ice (100 ml) and the mixture extracted with diethylether (3×). The organic phase was dried (Na₂SO₄) and concentrated to give 80.0 g (83%) of yellow oil.

¹H-NMR (CDCl₃): 7.79 (d, J=8.2, 2H); 7.35 (d, J=7.6, 2H); 4.16 (t, J=5.2, 2H); 3.69 (t, J=3.7, 2H); 3.63-3.58 (m, 2H); 3.59 (s, 4H); 3.55-3.51 (m, 2H); 3.37 (s, 3H); 2.45 (s, 3H).

¹³C-NMR (CDCl₃): 144.84 (s); 132.98 (s); 129.84 (d); 127.96 (d); 71.88 (t); 70.71 (t); 70.51 (t, 2×); 69.28 (t); 68.65 (t); 58.99 (q); 21.63 (q).

### b) Synthesis of 2-[2-(2-methoxyethoxy)ethoxy]ethyl-1H-isoindole-1,3(2H)-dione

A solution of 2-hydroxy-1,3-isoindoledione (10.40 g, 63.8 mmol), 2-[2-methoxyethoxy)ethoxy]ethyl 4-methylbenzenesulfonate (20.00 g, 62.8 mmol) and DBU (9.6 ml, 64.3 mmol) in DMF (200 ml) was heated at 80° C. for 16 h. The reaction mixture was concentrated, taken up in dichloromethane and washed with a saturated solution of NaHCO₃ (3×). Drying (Na₂SO₄) and concentrating gave 22.10 g of a yellow oil as the crude reaction product, which was used without further purification for the next reaction step.

### c) Synthesis of O-[2-[2-(2-methoxyethoxy)ethoxy]ethyl]hydroxylamine

Hydrazine hydrate (51% in water, 2.0 ml, 32.7 mmol) was added to a solution of 2-[2-(2-methoxyethoxy)ethoxy]ethyl-1H-isoindole-1,3(2H)-dione (11.11 g, 35.9 mmol) in methanol (125 ml). After stirring at room temperature for 14 h, the reaction mixture was concentrated and taken up in diethylether. A white solid was filtrated off and washed with ether before an aqueous solution of HCl (0.1 M, 20 ml) was added to the ether phase. After phase separation, the pH of the aqueous phase was adjusted to 10 by addition of an aqueous solution of NaOH (10%). After addition of diethylether (100 ml) and extraction, the organic layer was dried and concentrated to give 2.24 g (35%) of a yellow oil.

¹H-NMR (CDCl₃): 5.23 (br. s, 2H); 3.81-3.85 (m, 2H); 3.60-3.70 (m, 8H); 3.54-3.57 (m, 2H); 3.38 (s, 3H).

¹³C-NMR (CDCl₃): 74.81 (t); 71.95 (t); 70.60 (t); 70.53 (t, 2×); 69.61 (t); 59.03 (q).

### Preparation of 1-(aminooxy)-2-{2-[2-(aminooxy)ethoxy]ethoxy}ethane

### a) Synthesis of 2,2′-[1,2-ethanediylbis(oxy-2,1-ethanediyloxy)]bis(1H-isoindole-1,3(2H)-dione)

A solution of 2-hydroxy-1,3-isoindoledione (4.26 g, 26.1 mmol), 1,2-ethanediylbis(oxy-2,1-ethanediyl)bis(4-methylbenzenesulfonate) (6.00 g, 13.1 mmol) and DBU (3.97 g, 26.1 mmol) in DMF (290 ml) was heated at 80° C. for 20 h. The reaction mixture was concentrated, taken up in ethyl acetate and washed with a sat. solution of NaHCO₃ (3×) until the organic phase became colorless. Drying (Na₂SO₄) and concentrating gave 5.38 g (94%) of a slightly yellow solid.

¹H-NMR: 7.88-7.83 (m, 8H); 4.27-4.22 (m, 4H); 3.70-3.65 (m, 4H); 3.47 (s, 4H).

¹³C-NMR: 163.02 (s); 134.64 (d); 128.50 (s); 123.10 (d); 76.51 (t); 69.67 (t); 68.59 (t).

### b) Synthesis of 1-(aminooxy)-2-{2-[2-(aminooxy)ethoxy]ethoxy}ethane

Hydrazine hydrate (51% in water, 1.25 ml=1.28 g, 20.1 mmol) was added to a suspension of 2,2′-[1,2-ethanediylbis(oxy-2,1-ethanediyloxy)]bis(1H-isoindole-1,3(2H)-dione) (4.50 g, 10.2 mmol) in methanol (150 ml). The reaction mixture turned orange and the formation of a precipitate was observed. After stirring at room temperature for 4 h, the reaction mixture was filtered. Concentration of the filtrate and re-filtration (2×) gave a total of 1.54 g (84%) of a yellow oil.

¹H-NMR: 5.98 (s br., 4H); 3.65-3.59 (m, 4H); 3.56-3.46 (m, 8H).

¹³C-NMR: 74.07 (t); 69.68 (t); 68.49 (t).

Non commercial oxime derivatives were prepared as follows:

### Preparation of Benzaldehyde O-Octyloxime

A solution of O-octylhydroxylamine (1.00 g, 6.9 mmol) and benzaldehyde (1.10 g, 10.3 mmol) in ethanol (40 ml) was heated under reflux for 4 h. After cooling to room temperature, the mixture was concentrated and the excess of benzaldehyde was removed by Kugelrohr distillation (2×, 80° C., 5 mbar and 1 mbar) to give 0.96 g (60%) of a yellow oil.

¹H-NMR (CDCl₃): 8.07 (s, 1H); 7.61-7.54 (m, 2H); 7.41-7.32 (m, 3H); 4.16 (t, J=6.9, 2 H); 1.76-1.66 (m, 2H); 1.45-1.19 (m, 10H); 0.88 (t, J=6.9, 3H).

¹³C-NMR (CDCl₃): 148.18 (d); 132.52 (s); 129.62 (d); 128.65 (d); 126.94 (d); 74.45 (t); 31.84 (t); 29.43 (t); 29.26 (t); 29.16 (t); 25.94 (t); 22.67 (t); 14.11 (q).

### Preparation of Benzaldehyde O-Benzyloxime

A solution of O-benzylhydroxylamine hydrochloride (3.51 g, 22.0 mmol) and benzaldehyde (3.50 g, 33.0 mmol) in ethanol (50 ml) was heated at 60° C. for 3 h. After cooling to room temperature, the mixture was concentrated and the excess of benzaldehyde was removed by Kugelrohr distillation (65° C., 6-10 mbar) to give 4.54 g (98%) of a yellow oil.

¹H-NMR: 8.32 (s, 1H), 7.58-7.64 (m, 2H), 7.30-7.43 (m, 8H), 5.18 (s, 2H).

¹³C-NMR: 149.11 (d), 137.55 (s) 131.81 (s), 129.90 (d), 128.72 (d), 128.25 (d), 128.15 (d), 127.73 (d), 126.80 (d), 75.38 (t).

### Preparation of Benzaldehyde O-Trityloxime

A solution of O-(triphenylmethyl)hydroxylamine (1.00 g, 3.6 mmol) and benzaldehyde (0.60 g, 5.7 mmol) in ethanol (10 ml) was heated at 60° C. for 3 h. After cooling to room temperature and keeping at 4° C. overnight, the mixture was filtered to give 1.19 g (90%) of a white solid.

¹H-NMR: 8.47 (s, 1H); 7.50-7.40 (m, 2H); 7.38-7.32 (m, 15H), 7.3-7.25 (m, 3H).

¹³C-NMR: 149.24 (d); 143.93 (s); 132.03 (s); 129.86 (d); 128.66 (d); 128.61 (d); 127.57 (d); 127.06 (d); 126.76 (d); 90.21 (s).

### Preparation of benzaldehyde O-[2-(trimethylsilyl)ethyl]oxime

A solution of O-(2-trimethylsilylethyl)hydroxylamine hydrochloride (0.77 g, 4.5 mmol) and benzaldehyde (0.76 g, 7.2 mmol) in ethanol (10 ml) was heated at 60° C. for 3 h. After cooling to room temperature and keeping at 4° C. overnight, the mixture was concentrated and the remaining benzaldehyde distilled off (6-10 mbar, 65° C., 20 min) to give 1.19 g (93%) of a partially crystallised yellow oil.

¹H-NMR: 8.18 (s, 1H); 7.55-7.65 (m, 2H); 7.35-7.45 (m, 3H); 4.18 (t, J=8.2, 2H); 1.01 (t, J=8.2, 2H), 0.02 (s, 9H).

¹³C-NMR: 148.07 (d); 132.17 (s); 129.70 (d); 128.71 (d); 126.70 (d); 71.02 (t), 17.18 (t); −1.35 (q).

### Preparation of benzaldehyde O-(2-methoxyethyl)oxime

A suspension of O-(2-methoxyethyl)hydroxylamine hydrochloride (0.20 g, 1.6 mmol), K₂CO₃ (0.50 g), Na₂SO₄ (0.50 g) and benzaldehyde (0.25 g, 2.4 mmol) in ethanol (2 ml) was stirred at room temperature for 5 days. The mixture was concentrated, taken up in diethylether (20 ml) and washed with water (10 ml, 2×). Then the solvent was removed and the residue dried (0.1 mbar, 2 h) to give 0.15 g (53%) of yellow oil.

¹H-NMR: 8.26 (s, 1H); 7.59-7.64 (m, 2H); 7.40-7.43 (m, 3H); 4.22 (t, J=4.7, 2H); 3.59 (t, J=4.7, 2H); 3.28 (s, 3H).

¹³C-NMR: 148.74 (d); 131.87 (s); 129.83 (d); 128.70 (d); 126.75 (d); 72.78 (t); 70.13 (t); 58.02 (q).

### Use of Active Aldehydes or Ketones

The following examples illustrate the formation of dynamic mixtures using perfuming or flavoring ingredients as active aldehydes or ketones. However, they are also representative for the generation of dynamic mixtures according to the present invention in which the active aldehydes or ketones are useful as insect repellants or attractants, or as bactericides or fungicides. Some of the compounds described in the following examples, such as benzaldehyde, decanal, 3,7-dimethyl-6-octenal (citronellal), 2-furancarbaldehyde (furfural), 2-pentyl-1-cyclopentanone (Delphone) or 10-undecenal, are also known to be insect attractants or repellents (see for example: A. M. El-Sayed, The Pherobase 2005, http://www.pherobase.net) and/or to be active against bacteria (see for example: WO 01/24769 or EP 1 043 968).

### Example 1

### Formation of an Invention's Dynamic Mixture

The formation of the dynamic mixture was monitored by ¹H-NMR spectroscopy in buffered aqueous solution (DMSO-d₆/D₂O 2:1 (v/v)). The aqueous part of the acidic deuterated buffer stock solution was prepared from the following product quantities:

| | |
|---|---|
| Ortho-phosphoric acid (origin: Fluka) | 2.11 g |
| KH₂PO₄ (monobasic, origin: Acros) | 1.46 g |
| D₂O (origin: Euriso-top) | 110.51 g (=100 ml) |

Addition of 1.0 ml of DMSO-d₆ to 0.5 ml of the aqueous part of the buffer stock solution to give the final reaction solution for which a pH of ca. 4.5 (±0.5) was measured (pH-Indikatorstäbchen 0-6 Acilit®, origin: Merck).

To verify the formation of the same equilibrium for the formation and hydrolysis of oxime derivatives according to the present invention 180 mM solutions of a hydroxylamine derivative, an active aldehyde or ketone and the corresponding oxime derivative were prepared in DMSO-d₆, respectively. To 0.3 mL of the aqueous part of the deuterated buffer stock solution in an NMR tube were then added either 0.05 mL of the solution with the hydroxylamine derivative, 0.05 mL of the solution with the active aldehyde or ketone and 0.5 mL of DMSO-d₆ or, alternatively, 0.05 mL of the corresponding oxime derivative and 0.55 mL of DMSO-d₆, respectively. Each tube thus contains a mixture of DMSO-d₆/D₂O 2:1 (v/v). The NMR tubes were sonicated for 1 h and then left equilibrating at room temperature for 2 days before recording the ¹H-NMR spectra of the samples. For each sample the amount of free active aldehyde or ketone with respect to the amount of the oxime derivative was determined by integration of the corresponding signals. Another NMR measurement after 4 days showed that the equilibrium did not change. Depending on whether the dynamic mixture is obtained by using either the hydroxylamine or its corresponding hydrochloride salt, together with the active compound, slight modifications of the medium may occur and therefore different equilibria may be obtained.

The following amounts of free active aldehydes or ketones were detected from the sample containing the hydroxylamine derivative together with the aldehyde or ketone as compared to the reference sample containing the corresponding oxime derivative after 2 days:

| | Amount of |
|---|---|
| | free active |
| | aldehyde |
| Equilibrated dynamic mixture in DMSO-d₆/D₂O 2:1 (v/v) obtained from | or ketone ^{a}) |
| O-octylhydroxylamine and benzaldehyde | <5% |
| benzaldehyde O-octyloxime | <5% |
| O-octylhydroxylamine and furfural | 38% |
| O-octylhydroxylamine and decanal | 14% |
| O-octylhydroxylamine and Vertral ® | 38% |
| O-(cyclohexyl)hydroxylamine and benzaldehyde | 11% |
| O-(2-phenoxyethyl)hydroxylamine hydrochloride and Trifernal ® | <5% |
| O-(2-phenylethyl)hydroxylamine hydrochloride and benzaldehyde | <5% |
| O-(2-phenylethyl)hydroxylamine hydrochloride and 1,8-p-menthadien-7-al | 6% |
| O-benzylhydroxylamine hydrochloride and benzaldehyde | <5% |
| benzaldehyde O-benzyloxime | <5% |
| 2-(aminooxy)indane and furfural | <5% |
| O-(2-methoxyethyl)hydroxylamine hydrochloride and benzaldehyde | 85% |
| O-(2-methoxyethyl)hydroxylamine hydrochloride and furfural | 74% |
| O-(2-methoxyethyl)hydroxylamine hydrochloride and decanal | 12% |
| O-(2-methoxyethyl)hydroxylamine hydrochloride and Trifernal ® | 8% |
| O-(2-methoxyethyl)hydroxylamine hydrochloride and Delphone | 5% |
| O-(2-methoxyethyl)hydroxylamine hydrochloride and Vertral ® | 26% |
| O-(2-methoxyethyl)hydroxylamine hydrochloride and 1,8-p-menthadien-7-al | 95% |
| O-{2-[2-(2-methoxyethoxy)ethoxy]ethyl}hydroxylamine and benzaldehyde | 37% |
| 1-(aminooxy)-2-{2-[2-(aminooxy)ethoxy]ethoxy}ethane and benzaldehyde | 27% |
| 1-(aminooxy)-2-{2-[2-(aminooxy)ethoxy]ethoxy}ethane and furfural | 27% |
| 1-(aminooxy)-2-{2-[2-(aminooxy)ethoxy]ethoxy}ethane and Vertral ® | 9% |
| 1-(aminooxy)-2-{2-[2-(aminooxy)ethoxy]ethoxy}ethane and 1,8-p- | 50% |
| menthadien-7-al | |
| O-phenylhydroxylamine hydrochloride and benzaldehyde | 5% |
| carboxymethoxylamine hemihydrochloride and benzaldehyde | <5% |
| O-(2-trimethylsilylethyl)hydroxylamine hydrochloride and benzaldehyde | 7% |
| benzaldehyde O-[2-(trimethylsilyl)ethyl]oxime | 11% |
| O-(2-trimethylsilylethyl)hydroxylamine hydrochloride and furfural | 10% |
| O-(triphenylmethyl)hydroxylamine and benzaldehyde ^{b}) | <5% |
| benzaldehyde O-trityloxime ^{b}) | <5% |
| triphenylmethanesulfenamide and furfural ^{b}) | 50% |
| triphenylmethanesulfenamide and Trifernal ® ^{b}) | 14% |
| triphenylmethanesulfenamide and Vertral ® ^{b}) | 34% |
| triphenylmethanesulfenamide and 1,8-p-menthadien-7-al ^{b}) | 61% |

^{a}) the sum of the amount of free active aldehyde or ketone (=the amount of hydroxylamine derivative) and the corresponding oxime is 100%. ^{b}) measurements carried out at 2.25 mM in DMSO-d₆/D₂O 3:1 (v/v).

The data show for the reactions carried out in both directions that within the experimental error (ca. 1-2%) the same amount of free active aldehyde or ketone (and thus the same equilibrium) is reached for a dynamic mixture obtained either by reversible reaction of a hydroxylamine derivative with an active aldehyde or ketone in a water-containing medium or, alternatively, by hydrolysis of the corresponding oxime derivative. A low value of free active aldehyde or ketone furthermore indicates an increased effect of stabilisation of the compound in the aqueous medium as the labile carbonyl function is protected in the form of an oxime.

### Example 2

### Performance of a Softener Base Comprising an Invention's Dynamic Mixture

The use as perfuming ingredient of the present invention's mixture has been tested in a fabric softener. A fabric softener base with the following final composition has been prepared:

| | |
|---|---|
| Stepantex ® VK90 (origin: Stepan) | 16.5% by weight |
| Calcium chloride | 0.2% by weight |
| Water | 83.3% by weight |

The perfuming performance, over time, of the free perfuming aldehydes/ketones and of the invention's mixtures (i.e. the free perfuming aldehydes/ketones with an hydroxylamine derivative as additive) was determined in the following experiment:

1.80 g of the above fabric softener base were weighed into two small vials, respectively. Then 1 ml of a solution containing equimolar amounts (0.41 mmol) of 3,5,5-trimethylhexanal (58.1 mg), (R)-3,7-dimethyl-6-octenal (citronellal, 63.1 mg), decanal (63.6 mg), 4-phenyl-2-butanone (benzylacetone, 60.4 mg) 10-undecenal (68.7 mg) and (±)-exo-tricyclo[5.2.1.0(2,6)]decane-8exo-carbaldehyde (Vertral®, 67.3 mg) in 10 ml of ethanol was added to each vial. Then, 1 ml of a solution containing 355.8 mg (2.45 mmol) of O-octylhydroxylamine in 10 ml of ethanol was added to one of the samples, and 1 ml of ethanol was added to the other sample serving as the reference. The two samples were closed and left standing at room temperature to equilibrate. After 5 d, the samples were dispersed in a beaker with 600 ml of demineralized cold tap water, respectively. One cotton towel (EMPA cotton test cloth Nr. 221, origin: Eidgenössische Materialprüfanstalt (EMPA), pre-washed with an unperfumed detergent powder and cut to ca. 12×12 cm sheets) was added to each beaker and agitated manually for 3 min, left standing for 2 min, then wrung out by hand and weighed to obtain a constant quantity of residual water. The two towels were left drying overnight and analyzed the next day. Each towel was put into a headspace sampling cell (160 ml) thermostated at 25° C. and exposed to a constant air flow of ca. 205 ml/min, respectively. The air was filtered through active charcoal and aspirated through a saturated solution of NaCl (to ensure a constant humidity of the air of ca. 75%). During 15 min the headspace system was left equilibrating, then the volatiles were adsorbed during 15 min on a clean Tenax® cartridge. The sampling was repeated 7 times every hour. The cartridges were desorbed on a Perkin Elmer TurboMatrix ATD desorber coupled to a Carlo Erba MFC 500 gas chromatograph equipped with a J&W Scientific DB1 capillary column (30 m, i.d. 0.45 mm, film 0.42 μm) and a FID detector. The volatiles were analyzed using a two step temperature gradient starting from 70° C. to 130° C. at 3° C./min and then going to 260° C. at 25° C./min. The injection temperature was at 240° C., the detector temperature at 260° C. Headspace concentrations (in ng/l) were obtained by external standard calibrations of the corresponding fragrance aldehydes and ketones using ethanol solutions of five different concentrations. 0.2 μl of each calibration solution was injected onto Tenax® cartridges, which were immediately desorbed under the same conditions as those resulting from the headspace sampling.

The following amounts of aldehydes and ketones were detected from the sample containing the O-substituted hydroxylamine derivative as compared to the reference sample without the hydroxylamine derivative (in brackets):

| | 90 min | 210 min | 330 min | 450 min |
|---|---|---|---|---|
| | [ng/l] | [ng/l] | [ng/l] | [ng/l] |
| 3,5,5-Trimethyl- | 2.2 (2.0) | 2.0 (1.55) | 2.3 (1.6) | 2.1 (1.7) |
| hexanal | | | | |
| Citronellal | 0.9 (0.4) | 1.3 (0.4) | 1.2 (0.4) | 1.2 (0.0) |
| Decanal | 3.3 (3.2) | 4.6 (4.2) | 4.7 (3.9) | 5.4 (4.1) |
| Benzylacetone | 0.5 (0.0) | 0.4 (0.0) | 0.3 (0.0) | 0.5 (0.0) |
| 10-Undecenal | 2.0 (4.8) | 5.5 (8.5) | 6.0 (8.7) | 6.4 (8.9) |
| Vertral ® | 0.9 (0.9) | 1.6 (1.2) | 2.0 (1.6) | 2.5 (1.5) |

With the exception of 10-undecenal the headspace concentrations of the aldehydes and ketones were found to be higher in the presence of the hydroxylamine derivative than in its absence. The presence of the hydroxylamine has thus a positive effect on the long-lastingness of the fragrance perception on dry fabric.

### Example 3

### Performance of a Softener Base Comprising an Invention's Dynamic Mixture

1.80 g of the above fabric softener base (Example 2) were weighed into two vials respectively. Then 1 ml of benzaldehyde (39.6 mg, 0.37 mmol) in 5 ml of ethanol and O-(2-methoxyethyl)hydroxylamine hydrochloride (47.6 mg, 0.37 mmol) in 5 ml of ethanol were added to one of the vials; then 1 ml of the benzaldehyde solution and 1 ml of ethanol were added to the other vial serving as reference. The two samples were closed and left standing at room temperature to equilibrate for 5 days. After 5 days, the samples were dispersed in a beaker with 600 ml of demineralized cold tap water, respectively. Two cotton towels (EMPA cotton test cloth Nr. 221, origin: Eidgenössische Materialprüfanstalt (EMPA), pre-washed with an unperfumed detergent powder and cut to ca. 12×12 cm sheets) were added to each beaker and agitated manually for 3 min, left standing for 2 min, then wrung out by hand and weighed to obtain a constant quantity of residual water. The four towels were left drying overnight and analyzed pairwise (one sample with the hydroxylamine hydrochloride and one without) either the next day or after 3 days, respectively. Each towel was put into a headspace sampling cell (160 ml) thermostated at 25° C. and exposed to a constant air flow of ca. 200 ml/min. The air was filtered through active charcoal and aspirated through a saturated solution of NaCl (to ensure a constant humidity of the air of ca. 75%). During 15 min the headspace system was left equilibrating, then the volatiles were adsorbed during 15 min on a clean Tenax® cartridge. The sampling was repeated 7 times every hour. The cartridges were desorbed on a Perkin Elmer TurboMatrix ATD 350 desorber coupled to a Perkin Elmer Autosystem XL gas chromatograph equipped with a J&W Scientific DB1 capillary column (30 m, i.d. 0.25 mm, film 0.25 μm) and a Perkin Elmer Turbomass Upgrade mass spectrometer. The volatiles were analyzed using a two steps temperature gradient starting from 70° C. to 130° C. at 3° C./min and then going to 260° C. at 25° C./min. Headspace concentrations (in ng/l) were obtained by external standard calibrations using five different concentrations of benzaldehyde in ethanol and injecting 0.1, 0.2 or 0.3 μl of these calibration solutions onto Tenax® cartridges, respectively. The cartridges were then desorbed under the same conditions as those resulting from the headspace sampling.

The following amounts of benzyldehyde were detected from the samples containing the O-substituted hydroxylamine derivative as compared to the reference sample (in brackets) after 1 or 3 days:

| | 90 min | 210 min | 330 min | 450 min |
|---|---|---|---|---|
| | [ng/l] | [ng/l] | [ng/l] | [ng/l] |
| Benzaldehyde 1 day | 44.1 (1.3) | 35.1 (2.0) | 26.8 (1.5) | 22.2 (1.3) |
| Benzaldehyde 3 days | 25.0 (2.8) | 20.8 (0.0) | 14.7 (2.9) | 11.0 (1.2) |

The headspace concentrations of the benzaldehyde after 1 day and 3 days were found to be higher in the presence of the hydroxylamine derivative than in its absence. The presence of the hydroxylamine has thus a positive effect on the long-lastingness of the fragrance perception on dry fabric.

### Example 4

### Performance of a Softener Base Comprising an Invention's Dynamic Mixture

The experiment was carried out as described above (Example 2) by adding 44.1 mg of O-{2-[2-(2-methoxyethoxy)ethoxy]ethyl}hydroxylamine in 1 ml of ethanol and 1.80 g of the above fabric softener base into a small vial. To another vial, serving as the reference, 1 ml of ethanol and 1.80 g of the above fabric softener base were added. Then 1 ml of a solution containing equimolar amounts (0.41 mmol) of furfural (39.4 mg), citronellal (63.2 mg), Trifernal® (60.9 mg), delphone (63.2 mg) 10-undecenal (69.0 mg) and Vertral® (67.4 mg) in 10 ml of ethanol was added to both vials. The two samples were closed and left standing at room temperature to equilibrate. After 5 d, the samples were treated as described above (Example 2), using a constant air flow of ca. 200 ml/min and analyzed as described in Example 3. Headspace concentrations (in ng/l) were obtained by external standard calibrations of the corresponding fragrance aldehydes and ketones using ethanol solutions of five different concentrations. 0.1, 0.2 or 0.3 μl of these calibration solutions were injected onto Tenax® cartridges, respectively, and desorbed under the same conditions as those resulting from the headspace sampling.

The following amounts of aldehydes and ketones were detected from the sample containing the O-substituted hydroxylamine derivative as compared to the reference sample without the hydroxylamine derivative (in brackets):

| | 90 min | 210 min | 330 min | 450 min |
|---|---|---|---|---|
| | [ng/l] | [ng/l] | [ng/l] | [ng/l] |
| Furfural | 2.7 (1.0) | 1.4 (0.3) | 0.8 (0.0) | 0.7 (0.2) |
| Citronellal | 2.8 (1.5) | 2.7 (1.1) | 2.0 (1.0) | 1.6 (1.2) |
| Trifernal | 2.7 (1.0) | 7.2 (6.7) | 6.2 (5.9) | 5.2 (5.1) |
| Delphone | 1.1 (0.7) | 0.5 (0.4) | 0.3 (0.4) | 0.5 (0.4) |
| 10-Undecenal | 4.4 (12.3) | 6.7 (23.8) | 5.9 (21.6) | 4.7 (15.2) |
| Vertral ® | 2.1 (1.0) | 2.5 (1.4) | 2.1 (1.1) | 1.9 (0.9) |

With the exception of 10-undecenal the headspace concentrations of the aldehydes and ketones were found to be higher in the presence of the hydroxylamine derivative than in its absence. The presence of the hydroxylamine has thus a positive effect on the long-lastingness of the fragrance perception on dry fabric.

1.-14. (canceled)

15. A method to confer, enhance, improve or modify the odor properties of a perfuming composition or of a perfumed article, which method comprises adding to said composition or article an effective amount of a dynamic mixture, for the controlled release of active ketones or aldehydes, obtainable by reacting, in a water-containing medium,
i) at least one derivative of formula 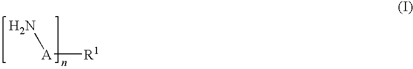
wherein:
A represents a functional group selected from the group consisting of the formulae (i) to (vi) 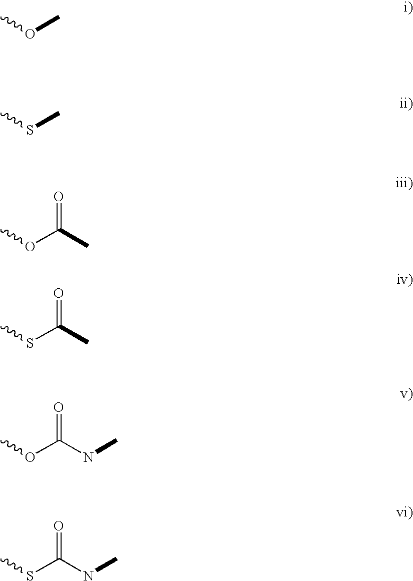
in which formulae the wavy line indicates the location of the bond between said NH₂ group and A, the bold lines indicate the location of the bond between said A and R′; and
I) n is an integer from 1 to 4; and
R¹ represents a trityl group or a mono-, di-, tri- or tetra-radical derived from a C₁ to C₁₈ linear, branched, cyclic or multicyclic hydrocarbon or polyethylene- or polypropylene-glycol group; said R¹ optionally comprising one, two, or three functional groups selected from the group consisting of silyl, ketone, amide, ether, thioether or secondary or tertiary amines; said R¹ being optionally substituted by up to three groups selected from the group consisting of NR²₂, NHR², (NR²R³₂)X, OR³, SO₃M, R³ and COOR⁴, R² representing a C₁ to C₁₀ alkyl, acyl or polyethylene- or polypropylene-glycol group, a phenyl group or a C₆ to C₉ alkylaryl group, and said R³ representing a hydrogen atom or a R² group, M representing a hydrogen atom or an alkali metal ion, R⁴ representing a M group or a R³ group and X representing a halogen atom or a sulphate; or
II) n is an integer varying from 2 to 5000; and R¹ represents a polyalkylene chain comprising between 2 and 5000 monomeric units;
with
ii) at least one active aldehyde or ketone having a molecular weight comprised between 80 and 230 g/mol and being a perfuming, flavoring, insect repellent or attractant, bactericide or fungicide ingredient.

16. The method according to claim 15, wherein the water-containing medium comprises at least 30% w/w of water.

17. The method according to claim 15, wherein the derivative of formula (I) is a compound wherein
A represents a functional group selected from the group of the formulae (i) or (ii), as defined above; and
I) n is an integer from 1 to 4; and
R¹ represents a trityl group or a mono-, di-, tri- or tetra-radical derived from a phenyl, benzyl or trityl group or from a C₁ to C₁₂ linear, branched, cyclic or bicyclic hydrocarbon or polyethylene- or polypropylene-glycol group, or a C₃-C₉ trialkyl amine; said R¹ optionally comprising one, two, or three functional groups selected from the group consisting of silyl, ketone, amide, ether, thioether or secondary or tertiary amines; said R¹ being optionally substituted by up to three groups selected from the group consisting of NR²₂, NHR², (NR²R³₂)X, OR³, SO₃M, R³ group and COOR⁴, R² representing a C₁ to C₁₀ alkyl, acyl or polyethylene- or polypropylene-glycol group, a phenyl group or a C₆ to C₉ alkylaryl group, and said R³ representing a hydrogen atom or a R² group, M representing a hydrogen atom or an alkali metal ion, R⁴ representing a M group or a R³ group and X representing a halogen atom or a sulphate; or
II) n is an integer varying from 2 to 5000; and R¹ represents a polyalkylene chain comprising between 2 and 5000 monomeric units.

18. The method according to claim 15, wherein the derivative of formula (I) is a compound of formula 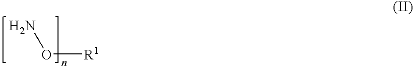
wherein
I) n is an integer from 1 to 4; and
R¹ represents a trityl group or a mono-, di-, tri- or tetra-radical derived from a phenyl, benzyl or trityl group or from a C₁ to C₁₂ linear, branched, cyclic or bicyclic hydrocarbon or polyethylene- or polypropylene-glycol group, or a C₃-C₉ trialkyl amine; said R¹ optionally comprising one, two, or three functional groups selected from the group consisting of silyl, ketone, amide, ether, thioether or secondary or tertiary amines; said R¹ being optionally substituted by up to three groups selected from the group consisting of NR²₂, NHR², (NR²R³₂)X, OR³, SO₃M, R³ group and COOR⁴, R² representing a C₁ to C₁₀ alkyl, acyl or polyethylene- or polypropylene-glycol group, a phenyl group or a C₆ to C₉ alkylaryl group, and said R³ representing a hydrogen atom or a R² group, M representing a hydrogen atom or an alkali metal ion, R⁴ representing a M group or a R³ group and X representing a halogen atom or a sulphate; or
II) n is an integer varying from 2 to 5000; and R¹ represents a polyalkylene chain having a molecular weight comprised between 48 and 80000.

19. The method according to claim 15, wherein the active aldehyde or ketone has a molecular weight comprised between 100 and 220 g/mol and is selected from the group consisting of an enal, an enone, an aldehyde comprising the moiety CH₂CHO or CHMeCHO, an aryl aldehyde or ketone and a cyclic or acyclic ketone.

20. The method according to claim 15, wherein the active aldehyde or ketone has a vapor pressure above 2.0 Pa.

21. The method according to claim 15, wherein the active aldehyde or ketone has a vapor pressure above 5.0 Pa.

22. A dynamic mixture, for the controlled release of active ketones or aldehydes, obtainable by reversibly reacting, in a water-containing medium,
i) at least one derivative of formula 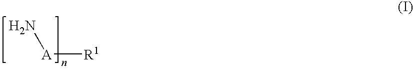
wherein:
A represents a functional group selected from the group consisting of the formulae (i) to (vi) 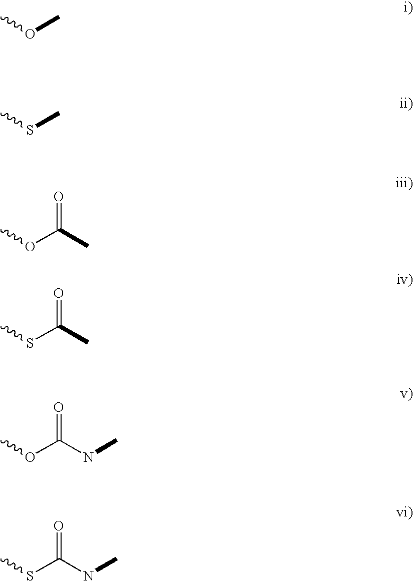
in which formulae the wavy line indicates the location of the bond between said NH₂ group and A, the bold lines indicate the location of the bond between said A and R¹; and
I) n is an integer from 1 to 4; and
R¹ represents a mono-, di-, tri- or tetra-radical derived from a
C₁ to C₁₈ linear, branched, cyclic or multicyclic hydrocarbon comprising one, two, or three functional groups selected from the group consisting of silyl, ketone, amide, ether, thioether or secondary or tertiary amines; said R¹ being optionally substituted by up to three groups selected from the group consisting of NR²₂, NHR², (NR²R³₂)X, OR³, SO₃M, R³ group and COOR⁴, R² representing a C₁ to C₁₀ alkyl, acyl or polyethylene- or polypropylene-glycol group, a phenyl group or a C₆ to C₉ alkylaryl group, and said R³ representing a hydrogen atom or a R² group, M representing a hydrogen atom or an alkali metal ion, R⁴ representing a M group or a R³ group and X representing a halogen atom or a sulphate;
C₁ to C₁₈ polyethylene- or polypropylene-glycol group; or
C₅ to C₁₈ linear, branched, cyclic or multicyclic alkyl or alkenyl group; or
a phenyl group, optionally substituted by up to three groups selected from the group consisting of NR²₂, NHR², (NR²R³₂)X, OR³, SO₃M, R³ group and COOR⁴, R² representing a C₁ to C₁₀ alkyl, acyl or polyethylene- or polypropylene-glycol group, a phenyl group or a C₆ to C₉ alkylaryl group, and said R³ representing a hydrogen atom or a R² group, M representing a hydrogen atom or an alkali metal ion, R⁴ representing a M group or a R³ group and X representing a halogen atom or a sulphate; or
a C₈ to C₂₀ linear, branched, cyclic or multicyclic hydrocarbon, optionally substituted by up to three groups selected from the group consisting of NR²₂, NHR², (NR²R³₂)X, OR³, SO₃M, R³ group and COOR⁴, R² representing a C₁ to C₁₀ alkyl, acyl or polyethylene- or polypropylene-glycol group, a phenyl group or a C₆ to C₉ alkylaryl group, and said R³ representing a hydrogen atom or a R² group, M representing a hydrogen atom or an alkali metal ion, R⁴ representing a M group or a R³ group and X representing a halogen atom or a sulphate;
II) n is an integer varying from 2 to 5000; and R¹ represents a polyalkylene chain comprising between 2 and 5000 monomeric units;
with
ii) at least one active aldehyde or ketone having a molecular weight comprised between 80 and 230 g/mol and being a perfuming, flavoring, insect repellent or attractant, bactericide or fungicide ingredient.

23. The dynamic mixture according to claim 22, wherein A represents a functional group of formula (I).

24. A compound of formula
R⁹ON═CR₂ (III)
wherein CR₂ represents a residue of the active aldehyde or ketone (O═CR₂) as above described, and R⁹ represents
a C₃-C₁₈ group of formula —(CH₂CH₂O)ₓH or —(CH₂CH₂O)_{w}CHR¹⁰₂, x representing an integer from 1 to 9, w representing an integer from 1 to 8, R¹⁰ representing, independently from each other, a hydrogen atom or a hydrocarbon group, optionally comprising one, two, or three functional groups selected from the group consisting of ketone, amide, ether, alcohol, or secondary or tertiary amines;
a group of formula —(CH₂CH₂O)_{w}ON═CR₂, CR₂ being defined as above w representing an integer from 1 to 9;
—(CH₂CH₂O)_{w}C₆H₅, w being as defined above;
—CH₂CH₂SiMe₃; or
—C₆H₁₁;
provided that cyclohexanone O—[2-(ethenyloxy)ethyl]oxime, 1-phenyl-ethanone O-[2-(ethenyloxy)ethyl]oxime, 1-phenyl-ethanone O-[2-[(tetrahydro-2H-pyran-2-yl)oxy]ethyl]oxime, 1-(4-methoxyphenyl)-ethanone O-[2-[(tetrahydro-2H-pyran-2-yl)oxy]ethyl]oxime, 4-phenyl-3-buten-2-one O-[2-[(4-methoxyphenyl)methoxy]ethyl]oxime, Benzaldehyde O—[2-(2-cyclohexen-1-yloxy)ethyl]oxime, 2,3-dihydro-1H-inden-1-one O-[2-[(tetrahydro-2H-pyran-2-yl)oxy]ethyl]oxime, 1-(1,3-benzodioxol-5-yl)-Ethanone O-[2-[(tetrahydro-2H-pyran-2-yl)oxy]ethyl]oxime, 1-(2-naphthalenyl)-ethanone O-[2-[(tetrahydro-2H-pyran-2-yl)oxy]ethyl]oxime, 2-naphthalenecarboxaldehyde O-[2-[(tetrahydro-2H-pyran-2-yl)oxy]ethyl]oxime, benzaldehyde O-[2-[(tetrahydro-2H-pyran-2-yl)oxy]ethyl]oxime, benzaldehyde O-(2-methoxyethyl)oxime, 4-methoxy-benzaldehyde O,O′-[1,2-ethanediylbis(oxy-2,1-ethanediyl)]dioxime, 2-hydroxy-benzaldehyde O,O′-[oxybis(2,1-ethanediyloxy-2,1-ethanediyl)]dioxime, 2-methoxy-benzaldehyde O,O′-[oxybis(2,1-ethanediyloxy-2,1-ethanediyl)]dioxime, 2-methoxy-benzaldehyde O,O′-(oxydi-2,1-ethanediyl)dioxime, 2-undecanone O-(26-hydroxy-3,6,9,12,15,18,21,24-octaoxahexacos-1-yl)oxime, 2-undecanone O-(17-hydroxy-3,6,9,12,15-pentaoxaheptadec-1-yl)oxime, 2-undecanone O-[2-[2-(2-hydroxyethoxy)ethoxy]ethyl]oxime, cyclohexanone O—[2-(oxiranyloxy)ethyl]oxime, benzaldehyde O—[2-(oxiranyloxy)ethyl]oxime and 2-undecanone O—[2-(2-hydroxyethoxy)ethyl]oxime are excluded.

25. A perfuming composition comprising:
i) as perfuming ingredient, a dynamic mixture as defined in claim 22;
ii) at least one ingredient selected from the group consisting of a perfumery carrier and a perfumery base; and
iii) optionally at least one perfumery adjuvant.

26. A perfumed article comprising:
i) as perfuming ingredient, a dynamic mixture as defined in claim 22; and
ii) a liquid consumer product base.

27. A perfumed article comprising:
i) a derivative of formula (I) or at least one O-alkyloxime obtainable from a derivative of formula (I) and a perfuming aldehyde or ketone as defined in claim 15, and a perfume or perfuming composition containing at least one perfuming aldehyde or ketone having a molecular weight comprised between 80 and 230 g/mol;
or
at least one O-alkyloxime obtainable from a derivative of formula (I) and an active aldehyde or ketone as defined in claim 15;
and
ii) a solid consumer product base intended to be used in the presence of water.

28. A method for prolonging the perfuming effect of a perfuming aldehyde or ketone, wherein at least one derivative of formula (I) as defined in claim 15 is included as an additive to a perfuming composition containing at least one perfuming aldehyde or ketone as defined in claim 15, and water.
